SEQUENCE LISTING (24 sequences)

SEQ ID NO: 1 - DNA, 3132 bp, Homo sapiens
  CDS (1)..(3132)
atgaagccaggctgtgcggcaggatctccagggaatgaatggattttcttcagcactgatgaaataaccacacgctataggaatacaatgtccaacgggggattgcaaagatctgtcatcctgtcagcacttattctgctacgagctgttactggattctctggagatggaagagctatatggtctaaaaatcctaattttactccggtaaatgaaagtcagctgtttctctatgacactttccctaaaaactttttctggggtattgggactggagcattgcaagtggaagggagttggaagaaggatggaaaaggaccttctatatgggatcatttcatccacacacaccttaaaaatgtcagcagcacgaatggttccagtgacagttatatttttctggaaaaagacttatcagccctggattttataggagtttctttttatcaattttcaatttcctggccaaggcttttccccgatggaatagtaacagttgccaacgcaaaaggtctgcagtactacagtactcttctggacgctctagtgcttagaaacattgaacctatagttactttataccactgggatttgcctttggcactacaagaaaaatatggggggtggaaaaatgataccataatagatatcttcaatgactatgccacatactgtttccagatgtttggggaccgtgtcaaatattggattacaattcacaacccatatctagtggcttggcatgggtatgggacaggtatgcatgcccctggagagaagggaaatttagcagctgtctacactgtgggacacaacttgatcaaggctcactcgaaagtttggcataactacaacacacatttccgcccacatcagaagggttggttatcgatcacgttgggatctcattggatcgagccaaaccggtcggaaaacacgatggatatattcaaatgtcaacaatccatggtttctgtgcttggatggtttgccaaccctatccatggggatggcgactatccagaggggatgagaaagaagttgttctccgttctacccattttctctgaagcagagaagcatgagatgagaggcacagctgatttctttgccttttcttttggacccaacaacttcaagcccctaaacaccatggctaaaatgggacaaaatgtttcacttaatttaagagaagcgctgaactggattaaactggaatacaacaaccctcgaatcttgattgctgagaatggctggttcacagacagtcgtgtgaaaacagaagacaccacggccatctacatgatgaagaatttcctcagccaggtgcttcaagcaataaggttagatgaaatacgagtgtttggttatactgcctggtctctcctggatggctttgaatggcaggatgcttacaccatccgccgaggattattttatgtggattttaacagtaaacagaaagagcggaaacctaagtcttcagcacactactacaaacagatcatacgagaaaatggtttttctttaaaagagtccacgccagatgtgcagggccagtttccctgtgacttctcctggggtgtcactgaatctgttcttaagcccgagtctgtggcttcgtccccacagttcagcgatcctcatctgtacgtgtggaacgccactggcaacagactgttgcaccgagtggaaggggtgaggctgaaaacacgacccgctcaatgcacagattttgtaaacatcaaaaaacaacttgagatgttggcaagaatgaaagtcacccactaccggtttgctctggattgggcctcggtccttcccactggcaacctgtccgcggtgaaccgacaggccctgaggtactacaggtgcgtggtcagtgaggggctgaagcttggcatctccgcgatggtcaccctgtattatccgacccacgcccacctaggcctccccgagcctctgttgcatgccgacgggtggctgaacccatcgacggccgaggccttccaggcctacgctgggctgtgcttccaggagctgggggacctggtgaagctctggatcaccatcaacgagcctaaccggctaagtgacatctacaaccgctctggcaacgacacctacggggcggcgcacaacctgctggtggcccacgccctggcctggcgcctctacgaccggcagttcaggccctcacagcgcggggccgtgtcgctgtcgctgcacgcggactgggcggaacccgccaacccctatgctgactcgcactggagggcggccgagcgcttcctgcagttcgagatcgcctggttcgccgagccgctcttcaagaccggggactaccccgcggccatgagggaatacattgcctccaagcaccgacgggggctttccagctcggccctgccgcgcctcaccgaggccgaaaggaggctgctcaagggcacggtcgacttctgcgcgctcaaccacttcaccactaggttcgtgatgcacgagcagctggccggcagccgctacgactcggacagggacatccagtttctgcaggacatcacccgcctgagctcccccacgcgcctggctgtgattccctggggggtgcgcaagctgctgcggtgggtccggaggaactacggcgacatggacatttacatcaccgccagtggcatcgacgaccaggctctggaggatgaccggctccggaagtactacctagggaagtaccttcaggaggtgctgaaagcatacctgattgataaagtcagaatcaaaggctattatgcattcaaactggctgaagagaaatctaaacccagatttggattcttcacatctgattttaaagctaaatcctcaatacaattttacaacaaagtgatcagcagcaggggcttcccttttgagaacagtagttctagatgcagtcagacccaagaaaatacagagtgcactgtctgcttattccttgtgcagaagaaaccactgatattcctgggttgttgcttcttctccaccctggttctactcttatcaattgccatttttcaaaggcagaagagaagaaagttttggaaagcaaaaaacttacaacacataccattaaagaaaggcaagagagttgttagc

SEQ ID NO: 2 - protein, 1044 aa, Homo sapiens
MKPGCAAGSPGNEWIFFSTDEITTRYRNTMSNGGLQRSVILSALILLRAVTGFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 3 - DNA, 2466 bp, homo sapiens
  CDS (1)..(2466)
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatcaccagaaaagatggaaaagaaattgcatgcagtgccggctgccaagacagtgaagttcaaatgcccttccagtgggacaccaaacccaacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgc

SEQ ID NO: 4 - protein, 822 aa, homo sapiens [sequence omitted]

SEQ ID NO: 5 - DNA, 2472 bp, Homo sapiens
  CDS (1)..(2472)
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatcaccagaaaagatggaaaagaaattgcatgcagtgccggctgccaagacagtgaagttcaaatgcccttccagtgggacaccaaacccaacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagcattcggggattaatagctcggatgcggaggtgctgaccctgttcaatgtgacagaggcccagagcggggagtatgtgtgtaaggtttccaattatattggtgaagctaaccagtctgcgtggctcactgtcaccagacctgtggcaaaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgc

SEQ ID NO: 6 - protein, 824 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKHSGINSSDAEVLTLFNVTEAQSGEYVCKVSNYIGEANQSAWLTVTRPVAKALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 7 - protein, 16 aa, Homo sapiens
HPIPDSSPLLQFGGQV

SEQ ID NO: 8 - protein, 7 aa, Homo sapiens
DDAQQTE

SEQ ID NO: 9 - DNA, 648 bp, Homo sapiens
  CDS (1)..(648)
atgcggagcgggtgtgtggtggtccacgtatggatcctggccggcctctggctggccgtggccgggcgtccacttgctttttctgatgctggtccacacgttcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaag

SEQ ID NO: 10 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 11 - DNA, 582 bp, Homo sapiens
  CDS (1)..(582)
cgtccacttgctttttctgatgctggtccacacgttcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaag

SEQ ID NO: 12 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 13 - DNA, 585 bp, Homo sapiens
  CDS (1)..(585)
atgcgtccacttgctttttctgatgctggtccacacgttcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaag

SEQ ID NO: 14 - protein, 195 aa, Homo sapiens
MRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 15 - DNA, 627 bp, Homo sapiens
  CDS (1)..(627)
atggactcggacgagaccgggttcgagcactcaggactgtgggtttctgtgctggctggtcttctgctgggagcctgccaggcacatccaattccagattcttctccattattacaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcc

SEQ ID NO: 16 - protein, 209 aa, Homo sapiens [sequence omitted]

SEQ ID NO: 17 - DNA, 543 bp, Homo sapiens
  CDS (1)..(543)
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcc

SEQ ID NO: 18 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 19 - DNA, 546 bp, Homo sapiens
  CDS (1)..(546)
atgcaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcacccccggagccacccggaatcctggccccccagccccccgatgtgggctcctcggaccctctgagcatggtgggaccttcccagggccgaagccccagctacgcttcc

SEQ ID NO: 20 - protein, 182 aa, Homo sapiens
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 21 - protein, 20 aa, Homo sapiens
RPLAFSDAGPHVHYGWGDPI

SEQ ID NO: 22 - protein, 8 aa, Homo sapiens
SGPHGLSS

SEQ ID NO: 23 - DNA, 570 bp, Homo sapiens
  CDS (1)..(570)
atgcatccaattccagattcttctccattattacaattcgggggccaagtccgcctgcggcacctgtacaccgatgatgcccagcagacagaatgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaag

SEQ ID NO: 24 - protein, 190 aa, Homo sapiens
MHPIPDSSPLLQFGGQVRLRHLYTDDAQQTECFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

(19) United States
(12) Patent Application Publication
(10) Pub. No.: US 2014/0189893 A1
(43) Pub. Date: Jul. 3, 2014

(54) METHOD OF IDENTIFYING COMPOUNDS THAT SPECIFICALLY MODULATE THE INTERACTION OF FGFR1 AND BETA KLOTHO

(71) Applicants: Yang Li, Mountain View, CA (US); Xinle Wu, San Diego, CA (US); Hongfei Ge, Foster City, CA (US); Helene Baribault, Redwood City, CA (US); Bryan Lemon, Mountain View, CA (US); Jackie Z. Sheng, Thousand Oaks, CA (US); Steven Vonderfecht, Azusa, CA (US); Jennifer Veronica Weiszmann, Orinda, CA (US); Jonitha Gardner, Hayward, CA (US); Grace Ki Jeong Lee, Simi Valley, CA (US)

(72) Inventors: Yang Li, Mountain View, CA (US); Xinle Wu, San Diego, CA (US); Hongfei Ge, Foster City, CA (US); Helene Baribault, Redwood City, CA (US); Bryan Lemon, Mountain View, CA (US); Jackie Z. Sheng, Thousand Oaks, CA (US); Steven Vonderfecht, Azusa, CA (US); Jennifer Veronica Weiszmann, Orinda, CA (US); Jonitha Gardner, Hayward, CA (US); Grace Ki Jeong Lee, Simi Valley, CA (US)

(73) Assignee: AMGEN INC., Thousand Oaks, CA (US)

(21) Appl. No.: 14/116,406

(22) PCT Filed: Feb. 20, 2012
(86) PCT No.: PCT/US12/25815

§ 371 Date: Jan. 31, 2014

Related U.S. Application Data
(60) Provisional application No. 61/484,585, filed on May 10, 2011.

(51) Int. Cl. G01N 33/74

(52) U.S. Cl. CPC G01N 33/74
USPC 800/3; 435/7.21; 435/7.4

(57) ABSTRACT

Methods of identifying compounds that specifically modulate the interaction of FGFR1 and β-Klotho are disclosed. Identified compounds can be useful in treating metabolic diseases and disorders that involve the interaction of FGFR1 and β-Klotho. In various embodiments the metabolic disease or disorder is diabetes, obesity, dyslipidemia, elevated glucose levels, elevated insulin levels and diabetic nephropathy.

This application claims the benefit of U.S. Provisional Appln. No. 61/484,585 filed May 10, 2011, which is incorporated by reference herein.

### REFERENCE TO THE SEQUENCE LISTING

The present application is being filed along with a Sequence Listing in electronic format. The Sequence Listing is provided as a file entitled A-1623-WO-PCT-Seq_Listing_ST25.txt, created Feb. 17, 2012, which is 96 KB in size. The information in the electronic format of the Sequence Listing is incorporated herein by reference in its entirety.

### FIELD OF THE INVENTION

The disclosed invention relates to a method of identifying compounds that specifically modulate the interaction of FGFR1 and β-Klotho. Such modulators can be useful to treat a metabolic disorder, such as diabetes, elevated glucose levels, elevated insulin levels, dyslipidemia, obesity or diabetic nephropathy.

### BACKGROUND OF THE INVENTION

Fibroblast Growth Factor 21 (FGF21) is a secreted polypeptide that belongs to a subfamily of Fibroblast Growth Factors (FGFs) that includes FGF19, FGF21, and FGF23 (Itoh et al., (2004) Trend Genet. 20:563-69). FGF21 is an atypical FGF in that it is heparin independent and functions as a hormone in the regulation of glucose, lipid, and energy metabolism.

It is highly expressed in liver and pancreas and is the only member of the FGF family to be primarily expressed in liver. Transgenic mice overexpressing FGF21 exhibit metabolic phenotypes of slow growth rate, low plasma glucose and triglyceride levels, and an absence of age-associated type 2 diabetes, islet hyperplasia, and obesity. Pharmacological administration of recombinant FGF21 protein in rodent and primate models results in normalized levels of plasma glucose, reduced triglyceride and cholesterol levels, and improved glucose tolerance and insulin sensitivity. In addition, FGF21 reduces body weight and body fat by increasing energy expenditure, physical activity, and metabolic rate. Experimental research provides support for the pharmacological administration of FGF21 for the treatment of diabetes, obesity, dyslipidemia, and other metabolic conditions or disorders in humans.

FGF21 is a liver derived endocrine hormone that stimulates glucose uptake in adipocytes and lipid homeostasis through the activation of its receptor. Interestingly, in addition to the canonical FGF receptor, the FGF21 receptor also comprises the membrane associated β-Klotho as an essential cofactor. Activation of the FGF21 receptor leads to multiple effects on a variety of metabolic parameters.

In mammals, FGFs mediate their action via a set of four FGF receptors, FGFR1-4, that in turn are expressed in multiple spliced variants, e.g., FGFR1c, FGFR2c, FGFR3c and FGFR4. Each FGF receptor contains an intracellular tyrosine kinase domain that is activated upon ligand binding, leading to downstream signaling pathways involving MAPKs (Erk1/2), RAF1, AKT1 and STATs. (Kharitonenkov et al., (2008) BioDrugs 22:37-44). Several reports suggested that the “c”-reporter splice variants of FGFR1-3 exhibit specific affinity to β-Klotho and could act as endogenous receptor for FGF21 (Kurosu et al., (2007) J. Biol. Chem. 282:26687-26695); Ogawa et al., (2007) Proc. Natl. Acad. Sci. USA 104:7432-7437); Kharitonenkov et al., (2008) J. Cell Physiol. 215:1-7). In the liver, which abundantly expresses both β-Klotho and FGFR4, FGF21 does not induce phosphorylation of MAPK albeit the strong binding of FGF21 to the β-Klotho-FGFR4 complex. In 3T3-L1 cells and white adipose tissue, FGFR1 is by far the most abundant receptor, and it is therefore most likely that FGF21's main functional receptors in this tissue are the β-Klotho-FGFR1c complexes.

The present disclosure provides the identity of the FGF21-mediated signaling complex. The present disclosure also provides a correlation and nexus between this complex and the treatment metabolic disorders, including diabetes, obesity and dyslipidemia.

### SUMMARY OF THE INVENTION

A method of identifying a compound that specifically modulates the interaction of FGFR1 and β-Klotho is provided. In one embodiment the method comprises: (a) determining a baseline level of FGFR1-mediated signaling in a signaling assay system comprising β-Klotho and FGFR1, wherein the FGFR1-mediated signal is one or more of Erk phosphorylation, FGFR1 phosphorylation and FRS2 phosphorylation; (b) contacting a test compound with the signaling assay system; (c) detecting a level of FGFR1-mediated signaling in the presence of the test compound; and (d) comparing the level of FGFR1-mediated signaling in the presence of the test compound with the baseline level of FGFR1-mediated signaling, wherein a difference between the two signaling levels indicates that the test compound modulates the interaction of FGFR1 and β-Klotho. In a one embodiment FGFR1 is FGFR1c, and other another embodiment FGFR1 is FGFR1b. In another embodiment the assay system comprises cells that express β-Klotho and FGFR1. In various embodiments the cells are human adipocyte cells, human liver cells or murine 3T3 adipocyte cells. In still other embodiments the assay system comprises one of a mouse model, a non-human primate model and a rat model. When the method is performed, it can be performed in the presence of a moiety that, in the presence of FGFR1 and β-Klotho, but in the absence of a test molecule, activates signaling; examples of such a moiety include one or more of FGF21, FGF19, a mutant form of FGF21, a mutant form of FGF19, an FGF21 analog, a FGF19 analog, an antibody and a peptibody.

### BRIEF DESCRIPTION OF THE DRAWINGS

FIG. 1 is a bar graph demonstrating the specific knockout of FGFR1 in the mice studied as determined using QPCR analysis.

FIG. 2 is a gel showing the effect of FGF19 and FGF21 on activation of Erk in adipocytes of FGFR1 knockout mice; FIG. 2A shows results obtained in adipose tissue of the mice and the FIG. 2B shows results obtained in liver tissue of the mice.

FIG. 3 is a schematic showing the study plan executed using the FGFR1 knockout mice.

FIG. 4 is a bar graph showing the effect of FGF19 and FGF21 on induced body weight reduction in DIO and the FGFR1 knockout mice over the two week period.

FIG. 5 is a series of plots showing the effect of FGF19 and FGF21 on induced OGTT improvement in DIO and the FGFR1 knockout mice over the two week period.

FIG. 6 is a bar graph showing the changes in weight of wild-type and FGFR1 knockout mice.

FIG. 7 is bar graph showing the effect of FGF19 and FGF21 on induction of glucose sensitivity of FGFR1 knockout mice.

FIG. 8 is plot showing the effect of FGF19 and FGF21 on induction of glucose sensitivity of FGFR1 knockout mice.

FIG. 9 is plot showing the effect of FGF19 and FGF21 on induction of glucose sensitivity of FGFR1 knockout mice.

FIG. 10 is plot showing the effect of FGF19 and FGF21 on induction of glucose sensitivity of FGFR1 knockout mice.

FIG. 11 is a schematic of construct FGF19-7, an FGF19 variant with receptor specificity biased toward FGFR1c.

FIG. 12 is a series of plots showing the activity of FGF19, FGF21 and FGF19-7 in a L6 transfection assay.

FIG. 13 is a plot showing the results of a glucose uptake assay using FGF19 and FGF19-7 in 3T3 cells.

FIG. 14 is two bar graphs and a plot showing the effect of FGF19 and FGF19-7 on insulin (FIG. 14A), triglycerides (FIG. 14B) and glucose (FIG. 14C).

FIG. 15 is two bar graphs showing the effect of FGF19 and FGF19-7 on body weight (FIG. 15A) and glucose levels (FIG. 15B) in a two week study using DIO mice.

FIG. 16 is a series of bar graphs and plots showing the effect of FGF19 and FGF19-7 on body weight, insulin and glucose in a two week study using ob/ob mice.

FIG. 17 is a series of bar graphs and plots showing the effect of FGF19 and FGF19-7 on body weight (FIG. 17A), glucose (FIG. 17B), triglycerides (FIG. 17C) and insulin (FIG. 17D) in a one year study in DIO mice; FIG. 17E shows the serum levels of FGF19 and FGF19-7.

### DETAILED DESCRIPTION OF THE INVENTION

The instant disclosure provides a method of identifying modulators (e.g., activators or inhibitors) of the FGFR1-mediated signaling pathway. The activation of this pathway can be beneficial for treating a metabolic disorder, such as diabetes, elevated glucose levels, elevated insulin levels, dyslipidemia or obesity, by administering to a subject in need thereof a therapeutically effective amount of a compound that actives the FGFR1-mediated pathway. Methods of administration and delivery are also provided.

Recombinant polypeptide and nucleic acid methods used herein, including in the Examples, are generally those set forth in Sambrook et al., Molecular Cloning: A Laboratory Manual (Cold Spring Harbor Laboratory Press, 1989) or Current Protocols in Molecular Biology (Ausubel et al., eds., Green Publishers Inc. and Wiley and Sons 1994), both of which are incorporated herein by reference for any purpose.

### I. GENERAL DEFINITIONS

Following convention, as used herein “a” and “an” mean “one or more” unless specifically indicated otherwise.

The term “β-Klotho polypeptide” also encompasses a β-Klotho polypeptide in which a naturally occurring β-Klotho polypeptide sequence (e.g., SEQ ID NO:2) has been modified. Such modifications include, but are not limited to, one or more amino acid substitutions, including substitutions with non-naturally occurring amino acids non-naturally-occurring amino acid analogs and amino acid mimetics and forms in which some or all of the membrane-spanning sequence of amino acids is removed, providing a soluble form of the protein.

In various embodiments, a β-Klotho polypeptide comprises an amino acid sequence that is at least about 85 percent identical to a naturally-occurring β-Klotho polypeptide (e.g., SEQ ID NO:2). In other embodiments, a β-Klotho polypeptide comprises an amino acid sequence that is at least about 90 percent, or about 95, 96, 97, 98, or 99 percent identical to a naturally-occurring β-Klotho polypeptide amino acid sequence (e.g., SEQ ID NO:2). Such β-Klotho polypeptides preferably, but need not, possess at least one activity of a wild-type β-Klotho polypeptide, such as the ability to lower blood glucose, insulin, triglyceride, or cholesterol levels; the ability to reduce body weight; or the ability to improve glucose tolerance, energy expenditure, or insulin sensitivity.

A β-Klotho polypeptide is preferably biologically active. In various respective embodiments, a β-Klotho polypeptide has a biological activity that is equivalent to, greater to or less than that of the naturally occurring form of the mature β-Klotho protein. Examples of biological activities include the ability to induce FGFR signaling, to lower blood glucose, insulin, triglyceride, or cholesterol levels; the ability to reduce body weight; or the ability to improve glucose tolerance, energy expenditure, or insulin sensitivity, when associated with FGFR1 and one of FGF19 and FGF21.

As used herein, the term “FGF19 polypeptide” refers to a polypeptide expressed in any species, including humans. For purposes of this disclosure, the term “FGF19 polypeptide” can be used interchangeably to refer to any full-length FGF19 polypeptide, e.g., SEQ ID NO:10, which consists of 216 amino acid residues and which is encoded by the nucleotide sequence of SEQ ID NO: 9, and any mature form of the polypeptide, e.g., SEQ ID NO:12, which consists of 194 amino acid residues and which is encoded by the nucleotide sequence of SEQ ID NO:11, and in which the 22 amino acid residues at the amino-terminal end of the full-length FGF 19 polypeptide (i.e., those residues which constitute the signal peptide) have been removed. A bacterially expressed form of a mature FGF19 polypeptide can be produced from the nucleotide of SEQ ID NO:13 and have the amino acid sequence of SEQ ID NO:14, and which will comprise an N-terminal methionine residue. A “FGF19 polypeptide” can be encoded by SEQ ID NOs:9, 11, and 13, for example, as well as any polynucleotide sequence that, due to the degeneracy of the genetic code, has a polynucleotide sequence that is altered by one or more bases from the polynucleotide sequences of SEQ ID NOs:9, 11 and 13, as well as allelic variants of SEQ ID NOs:9, 11 and 13. The term “FGF19 polypeptide” also encompasses naturally-occurring FGF19 variants. A “FGF19” polypeptide can but need not incorporate one or more non-naturally occurring amino acids.

As used herein, the term “FGF21 polypeptide” refers to a polypeptide expressed in any species, including humans. For purposes of this disclosure, the term “FGF21 polypeptide” can be used interchangeably to refer to any full-length FGF21 polypeptide, e.g., SEQ ID NO:16, which consists of 209 amino acid residues and which is encoded by the nucleotide sequence of SEQ ID NO:15; any mature form of the polypeptide, e.g., SEQ ID NO:18, which consists of 181 amino acid residues and which is encoded by the nucleotide sequence of SEQ ID NO:17, and in which the 28 amino acid residues at the amino-terminal end of the full-length FGF21 polypeptide (i.e., those residues which constitute the signal peptide) have been removed. A bacterially expressed form of a mature FGF21 polypeptide can be produced from the nucleotide of SEQ ID NO:20 and have the amino acid sequence of SEQ ID NO:19 and will comprise an N-terminal methionine residue. A “FGF21 polypeptide” can be encoded by SEQ ID NOs:15, 17 and 19, for example, as well as any polynucleotide sequence that, due to the degeneracy of the genetic code, has a polynucleotide sequence that is altered by one or more bases from the polynucleotide sequence of SEQ ID NOs: 15, 17 and 19, as well as allelic variants of SEQ ID NOs: 15, 17 and 19. The term “FGF21 polypeptide” also encompasses naturally-occurring variants, including the Leu/Pro SNP that is found at position 146 of the mature form of FGF21 and at position 174 of the full length form of FGF21. A “FGF21” polypeptide can but need not incorporate one or more non-naturally occurring amino acids.

The term “FGFR1” means a naturally-occurring wild-type Fibroblast Growth Factor Receptor 1, including splice forms 1b and 1c, polypeptide expressed in a mammal, such as a human or a mouse. For purposes of this disclosure, the term FGFR1 can be used interchangeably to refer to any FGFR1 polypeptide, e.g., SEQ ID NO:4, which consists of 822 amino acid residues and which is encoded by the nucleotide sequence SEQ ID NO:3 or SEQ ID NO:6, which consists of 824 amino acid residues and which is encoded by the nucleotide sequence SEQ ID NO:5. FGFR1 polypeptides can but need not comprise an amino-terminal methionine, which may be introduced by engineering or as a result of a bacterial expression process.

The term “FGFR1b” means a naturally-occurring wild-type Fibroblast Growth Factor Receptor, Splice form 1c, polypeptide expressed in a mammal, such as a human or a mouse. For purposes of this disclosure, the term FGFR1b can be used interchangeably to refer to any FGFR1b polypeptide, e.g., SEQ ID NO:6, which consist of 824 amino acid residues and which is encoded by the nucleotide sequence SEQ ID NO:5. FGFR1b polypeptides can but need not comprise an amino-terminal methionine, which may be introduced by engineering or as a result of a bacterial expression process.

The term FGFR1b also encompasses a FGFR1b polypeptide in which a naturally occurring FGFR1b polypeptide sequence (e.g., SEQ ID NO:6) has been modified. Such modifications include, but are not limited to, one or more amino acid substitutions, including substitutions with non-naturally occurring amino acids non-naturally-occurring amino acid analogs and amino acid mimetics and forms in which some or all of the membrane-spanning sequence of amino acids is removed, providing a soluble form of the protein.

In various embodiments, FGFR1b comprises an amino acid sequence that is at least about 85 percent identical to a naturally-occurring FGFR1b polypeptide (e.g., SEQ ID NO:5). In other embodiments, FGFR1b comprises an amino acid sequence that is at least about 90 percent, or about 95, 96, 97, 98, or 99 percent identical to a naturally-occurring FGFR1b amino acid sequence (e.g., SEQ ID NO:6). Such FGFR1b's preferably, but need not, possess at least one activity of a wild-type FGFR1b, such as the ability to lower blood glucose, insulin, triglyceride, or cholesterol levels; the ability to reduce body weight; and the ability to improve glucose tolerance, energy expenditure, or insulin sensitivity when associated with β-Klotho and FGF19 or FGF21.

FGFR1b is preferably biologically active. In various respective embodiments, FGFR1b has a biological activity that is equivalent to, greater to or less than that of the naturally occurring form of an FGFR1b. Examples of biological activities include the ability to induce FGFR signaling, to lower blood glucose, insulin, triglyceride, or cholesterol levels; the ability to reduce body weight; and the ability to improve glucose tolerance, energy expenditure, or insulin sensitivity when associated with β-Klotho and one of FGF19 and FGF21.

The term “FGFR1c” means a naturally-occurring wild-type Fibroblast Growth Factor Receptor, Splice form 1c, polypeptide expressed in a mammal, such as a human or a mouse. For purposes of this disclosure, the term FGFR1c can be used interchangeably to refer to any FGFR1c polypeptide, e.g., SEQ ID NO:4, which consist of 822 amino acid residues and which is encoded by the nucleotide sequence SEQ ID NO:3. FGFR1c polypeptides can but need not comprise an amino-terminal methionine, which may be introduced by engineering or as a result of a bacterial expression process.

The term FGFR1c also encompasses a FGFR1c polypeptide in which a naturally occurring FGFR1c polypeptide sequence (e.g., SEQ ID NO:4) has been modified. Such modifications include, but are not limited to, one or more amino acid substitutions, including substitutions with non-naturally occurring amino acids non-naturally-occurring amino acid analogs and amino acid mimetics and forms in which some or all of the membrane-spanning sequence of amino acids is removed, providing a soluble form of the protein.

In various embodiments, FGFR1c comprises an amino acid sequence that is at least about 85 percent identical to a naturally-occurring FGFR1c polypeptide (e.g., SEQ ID NO:4 and, in other embodiments, the sequences of NP_{—}001167534, NP_{—}001167535, NP_{—}001167536, NP_{—}001167537, NP_{—}001167538, NP_{—}075594, NP_{—}075598). In other embodiments, FGFR1c comprises an amino acid sequence that is at least about 90 percent, or about 95, 96, 97, 98, or 99 percent identical to a naturally-occurring FGFR1c amino acid sequence (e.g., SEQ ID NO:4). Such FGFR1c's preferably, but need not, possess at least one activity of a wild-type FGFR1c, such as the ability to lower blood glucose, insulin, triglyceride, or cholesterol levels; the ability to reduce body weight; or the ability to improve glucose tolerance, energy expenditure, or insulin sensitivity, when associated with β-Klotho and one of FGF21 and FGF19.

FGFR1c is preferably biologically active. In various respective embodiments, FGFR1c has a biological activity that is equivalent to, greater to or less than that of the naturally occurring form of an FGFR1c. Examples of biological activities include the ability to induce FGFR signaling, to lower blood glucose, insulin, triglyceride, or cholesterol levels; the ability to reduce body weight; or the ability to improve glucose tolerance, energy expenditure, or insulin sensitivity when associated with β-Klotho and FGF21.

As used herein a “conservative amino acid substitution” can involve a substitution of a native amino acid residue (i.e., a residue found in a given position of the wild-type β-Klotho polypeptide sequence) with a normative residue (i.e., a residue that is not found in a given position of the wild-type β-Klotho polypeptide sequence) such that there is little or no effect on the polarity or charge of the amino acid residue at that position. Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Naturally occurring residues can be divided into classes based on common side chain properties:

(1) hydrophobic: norleucine, Met, Ala, Val, Leu, Ile;

(2) neutral hydrophilic: Cys, Ser, Thr;

(3) acidic: Asp, Glu;

(4) basic: Asn, Gln, His, Lys, Arg;

(5) residues that influence chain orientation: Gly, Pro; and

(6) aromatic: Trp, Tyr, Phe.

Additional groups of amino acids can also be formulated using the principles described in, e.g., Creighton (1984) PROTEINS: STRUCTURE AND MOLECULAR PROPERTIES (2d Ed. 1993), W.H. Freeman and Company. In some instances it can be useful to further characterize substitutions based on two or more of such features (e.g., substitution with a “small polar” residue, such as a Thr residue, can represent a highly conservative substitution in an appropriate context).

Conservative substitutions can involve the exchange of a member of one of these classes for another member of the same class. Non-conservative substitutions can involve the exchange of a member of one of these classes for a member from another class.

Synthetic, rare, or modified amino acid residues having known similar physiochemical properties to those of an above-described grouping can be used as a “conservative” substitute for a particular amino acid residue in a sequence. For example, a D-Arg residue may serve as a substitute for a typical L-Arg residue. It also can be the case that a particular substitution can be described in terms of two or more of the above described classes (e.g., a substitution with a small and hydrophobic residue means substituting one amino acid with a residue(s) that is found in both of the above-described classes or other synthetic, rare, or modified residues that are known in the art to have similar physiochemical properties to such residues meeting both definitions).

As used herein, the term “FGFR1-mediated signaling” means activation of downstream signaling pathway typical of FGFR activation for example, receptor autophosphorylation, and/or phosphorylation of FRS2 and/or ERK. In addition, at a systemic level FGFR1-mediated signaling may lead to ability to lower blood glucose, insulin, triglyceride, or cholesterol levels; the ability to reduce body weight; or the ability to improve glucose tolerance, energy expenditure, or insulin sensitivity.

Methods for identifying and measuring FGFR1-mediated signaling include those assays provided herein, for example in Example 1. Other approaches to measuring FGFR1-mediated signaling include various methods of detecting the phosphorylation states of receptors or components of signaling cascade (e.g., FGFR1c and FGFR1b); such approaches include but are not limited to the use of various reagents that can recognize phosphorylated proteins such as antibodies, MS, or various separation methods. In addition, FGFR1-mediated signaling can be assessed by measuring body weight, blood parameters such as glucose, insulin, lipids, energy expenditure, insulin sensitivity, glucose uptake, and other parameters in vivo and/or in vitro. FGFR1-mediated signaling refers to any signaling determined as described herein that changes the end point relative to a predetermined background level in a particular assay system.

### II. β-KLOTHO AND FGFR1C POLYPEPTIDES AND NUCLEIC ACIDS

As disclosed herein, a β-Klotho polypeptide or an FGFR1c protein described by the instant disclosure can be engineered and/or produced using standard molecular biology methodology. In various examples, a nucleic acid sequence encoding a β-Klotho polypeptide or an FGFR1c protein, which can comprise all or a portion of SEQ ID NO:4 can be isolated and/or amplified from genomic DNA, or cDNA using appropriate oligonucleotide primers. Primers can be designed based on the nucleic and amino acid sequences provided herein according to standard (RT)-PCR amplification techniques. The amplified nucleic acid can then be cloned into a suitable vector and characterized by DNA sequence analysis.

Oligonucleotides for use as probes in isolating or amplifying all or a portion of the β-Klotho polypeptide or an FGFR1c protein sequences provided herein can be designed and generated using standard synthetic techniques, e.g., automated DNA synthesis apparatus, or can be isolated from a longer sequence of DNA.

### II.A. β-Klotho Polypeptide and Polynucleotide Sequences

In vivo, β-Klotho is expressed as a contiguous amino acid sequence comprising a signal sequence.

The amino acid sequence of full length human β-Klotho is:

SEQ ID NO: 2
MKPGCAAGSPGNEWIFFSTDEITTRYRNTMSNGGLQRSVILSALILLRAV
TGFSGDGRAIWSKNPNFTPVNESQLFLYDTFPKNFFWGIGTGALQVEGSW
KKDGKGPSIWDHFIHTHLKNVSSTNGSSDSYIFLEKDLSALDFIGVSFYQ
FSISWPRLFPDGIVTVANAKGLQYYSTLLDALVLRNIEPIVTLYHWDLPL
EKYGGWKNDTIIDIFNDYATYCFQMFGDRVKYWITIHNPYLVAWHGYGTA
LQGMHAPGEKGNLAAVYTVGHNLIKAHSKVWHNYNTHFRPHQKGWLSITL
GSHWIEPNRSENTMDIFKCQQSMVSVLGWFANPIHGDGDYPEGMRKKLFS
VLPIFSEAEKHEMRGTADFFAFSFGPNNFKPLNTMAKMGQNVSLNLREAL
NWIKLEYNNPRILIAENGWFTDSRVKTEDTTAIYMMKNFLSQVLQAIRLD
EIRVFGYTAWSLLDGFEWQDAYTIRRGLFYVDFNSKQKERKPKSSAHYYK
QIIRENGFSLKESTPDVQGQFPCDFSWGVTESVLKPESVASSPQFSDPHL
YVWNATGNRLLHRVEGVRLKTRPAQCTDFVNIKKQLEMLARMKVTHYRFA
LDWASVLPTGNLSAVNRQALRYYRCVVSEGLKLGISAMVTLYYPTHAHLG
LPEPLLHADGWLNPSTAEAFQAYAGLCFQELGDLVKLWITINEPNRLSDI
YNRSGNDTYGAAHNLLVAHALAWRLYDRQFRPSQRGAVSLSLHADWAEPA
NPYADSHWRAAERFLQFEIAWFAEPLFKTGDYPAAMREYIASKHRRGLSS
SALPRLTEAERRLLKGTVDFCALNHFTTRFVMHEQLAGSRYDSDRDIQFL
QDITRLSSPTRLAVIPWGVRKLLRWVRRNYGDMDIYITASGIDDQALEDD
RLRKYYLGKYLQEVLKAYLIDKVRIKGYYAFKLAEEKSKPRFGFFTSDFK
AKSSIQFYNKVISSRGFPFENSSSRCSQTQENTECTVCLFLVQKKPLIFL
GCCFFSTLVLLLSIAIFQRQKRRKFWKAKNLQHIPLKKGKRVVS,
(signal sequence underlined)

which is encoded by the DNA sequence

SEQ ID NO: 1
ATGAAGCCAGGCTGTGCGGCAGGATCTCCAGGGAATGAATGGATTTTCTT
CAGCACTGATGAAATAACCACACGCTATAGGAATACAATGTCCAACGGGG
GATTGCAAAGATCTGTCATCCTGTCAGCACTTATTCTGCTACGAGCTGTT
ACTGGATTCTCTGGAGATGGAAGAGCTATATGGTCTAAAAATCCTAATTT
TACTCCGGTAAATGAAAGTCAGCTGTTTCTCTATGACACTTTCCCTAAAA
ACTTTTTCTGGGGTATTGGGACTGGAGCATTGCAAGTGGAAGGGAGTTGG
AAGAAGGATGGAAAAGGACCTTCTATATGGGATCATTTCATCCACACACA
CCTTAAAAATGTCAGCAGCACGAATGGTTCCAGTGACAGTTATATTTTTC
TGGAAAAAGACTTATCAGCCCTGGATTTTATAGGAGTTTCTTTTTATCAA
TTTTCAATTTCCTGGCCAAGGCTTTTCCCCGATGGAATAGTAACAGTTGC
CAACGCAAAAGGTCTGCAGTACTACAGTACTCTTCTGGACGCTCTAGTGC
TTAGAAACATTGAACCTATAGTTACTTTATACCACTGGGATTTGCCTTTG
GCACTACAAGAAAAATATGGGGGGTGGAAAAATGATACCATAATAGATAT
CTTCAATGACTATGCCACATACTGTTTCCAGATGTTTGGGGACCGTGTCA
AATATTGGATTACAATTCACAACCCATATCTAGTGGCTTGGCATGGGTAT
GGGACAGGTATGCATGCCCCTGGAGAGAAGGGAAATTTAGCAGCTGTCTA
CACTGTGGGACACAACTTGATCAAGGCTCACTCGAAAGTTTGGCATAACT
ACAACACACATTTCCGCCCACATCAGAAGGGTTGGTTATCGATCACGTTG
GGATCTCATTGGATCGAGCCAAACCGGTCGGAAAACACGATGGATATATT
CAAATGTCAACAATCCATGGTTTCTGTGCTTGGATGGTTTGCCAACCCTA
TCCATGGGGATGGCGACTATCCAGAGGGGATGAGAAAGAAGTTGTTCTCC
GTTCTACCCATTTTCTCTGAAGCAGAGAAGCATGAGATGAGAGGCACAGC
TGATTTCTTTGCCTTTTCTTTTGGACCCAACAACTTCAAGCCCCTAAACA
CCATGGCTAAAATGGGACAAAATGTTTCACTTAATTTAAGAGAAGCGCTG
AACTGGATTAAACTGGAATACAACAACCCTCGAATCTTGATTGCTGAGAA
TGGCTGGTTCACAGACAGTCGTGTGAAAACAGAAGACACCACGGCCATCT
ACATGATGAAGAATTTCCTCAGCCAGGTGCTTCAAGCAATAAGGTTAGAT
GAAATACGAGTGTTTGGTTATACTGCCTGGTCTCTCCTGGATGGCTTTGA
ATGGCAGGATGCTTACACCATCCGCCGAGGATTATTTTATGTGGATTTTA
ACAGTAAACAGAAAGAGCGGAAACCTAAGTCTTCAGCACACTACTACAAA
CAGATCATACGAGAAAATGGTTTTTCTTTAAAAGAGTCCACGCCAGATGT
GCAGGGCCAGTTTCCCTGTGACTTCTCCTGGGGTGTCACTGAATCTGTTC
TTAAGCCCGAGTCTGTGGCTTCGTCCCCACAGTTCAGCGATCCTCATCTG
TACGTGTGGAACGCCACTGGCAACAGACTGTTGCACCGAGTGGAAGGGGT
GAGGCTGAAAACACGACCCGCTCAATGCACAGATTTTGTAAACATCAAAA
AACAACTTGAGATGTTGGCAAGAATGAAAGTCACCCACTACCGGTTTGCT
CTGGATTGGGCCTCGGTCCTTCCCACTGGCAACCTGTCCGCGGTGAACCG
ACAGGCCCTGAGGTACTACAGGTGCGTGGTCAGTGAGGGGCTGAAGCTTG
GCATCTCCGCGATGGTCACCCTGTATTATCCGACCCACGCCCACCTAGGC
CTCCCCGAGCCTCTGTTGCATGCCGACGGGTGGCTGAACCCATCGACGGC
CGAGGCCTTCCAGGCCTACGCTGGGCTGTGCTTCCAGGAGCTGGGGGACC
TGGTGAAGCTCTGGATCACCATCAACGAGCCTAACCGGCTAAGTGACATC
TACAACCGCTCTGGCAACGACACCTACGGGGCGGCGCACAACCTGCTGGT
GGCCCACGCCCTGGCCTGGCGCCTCTACGACCGGCAGTTCAGGCCCTCAC
AGCGCGGGGCCGTGTCGCTGTCGCTGCACGCGGACTGGGCGGAACCCGCC
AACCCCTATGCTGACTCGCACTGGAGGGCGGCCGAGCGCTTCCTGCAGTT
CGAGATCGCCTGGTTCGCCGAGCCGCTCTTCAAGACCGGGGACTACCCCG
CGGCCATGAGGGAATACATTGCCTCCAAGCACCGACGGGGGCTTTCCAGC
TCGGCCCTGCCGCGCCTCACCGAGGCCGAAAGGAGGCTGCTCAAGGGCAC
GGTCGACTTCTGCGCGCTCAACCACTTCACCACTAGGTTCGTGATGCACG
AGCAGCTGGCCGGCAGCCGCTACGACTCGGACAGGGACATCCAGTTTCTG
CAGGACATCACCCGCCTGAGCTCCCCCACGCGCCTGGCTGTGATTCCCTG
GGGGGTGCGCAAGCTGCTGCGGTGGGTCCGGAGGAACTACGGCGACATGG
ACATTTACATCACCGCCAGTGGCATCGACGACCAGGCTCTGGAGGATGAC
CGGCTCCGGAAGTACTACCTAGGGAAGTACCTTCAGGAGGTGCTGAAAGC
ATACCTGATTGATAAAGTCAGAATCAAAGGCTATTATGCATTCAAACTGG
CTGAAGAGAAATCTAAACCCAGATTTGGATTCTTCACATCTGATTTTAAA
GCTAAATCCTCAATACAATTTTACAACAAAGTGATCAGCAGCAGGGGCTT
CCCTTTTGAGAACAGTAGTTCTAGATGCAGTCAGACCCAAGAAAATACAG
AGTGCACTGTCTGCTTATTCCTTGTGCAGAAGAAACCACTGATATTCCTG
GGTTGTTGCTTCTTCTCCACCCTGGTTCTACTCTTATCAATTGCCATTTT
TCAAAGGCAGAAGAGAAGAAAGTTTTGGAAAGCAAAAAACTTACAACACA
TACCATTAAAGAAAGGCAAGAGAGTTGTTAGC,
(signal sequence underlined).

A β-Klotho sequence can also incorporate variant forms, including silent and coding single nucleotide polymorphisms such as those found at position 65 (Phe to Ala mutation), 166 (Val to Ala mutation), 728 (Arg to Gln mutation), 747 (Ala to Val), 906 (Tyr to His mutation) and 1020 (Gln to Lys mutation).

As stated herein, the term “β-Klotho polypeptide” refers to a β-Klotho polypeptide comprising the human amino acid sequences SEQ ID NO:2. The term “β-Klotho polypeptide,” however, also encompasses polypeptides comprising an amino acid sequence that differs from the amino acid sequence of a naturally occurring β-Klotho polypeptide sequence, e.g., SEQ ID NO:2, by one or more amino acids, such that the sequence is at least 85% identical to SEQ ID NO:2. β-Klotho polypeptides can be generated by introducing one or more amino acid substitutions, either conservative or non-conservative and using naturally or non-naturally occurring amino acids, at particular positions of the β-Klotho polypeptide.

Nucleic acid sequences encoding a β-Klotho polypeptide provided herein, including those degenerate to SEQ ID NO:1, and those encoding polypeptide variants of SEQ ID NO:2 form other aspects of the instant disclosure.

### II.B. FGFR1c Polypeptide and Polynucleotide Sequences

In vivo, FGFR1c is expressed as a contiguous amino acid sequence comprising a signal sequence. Variants of FGFR1c are known and form aspects of the term “FGFR1c” as used herein, including those comprising truncated N-termini relative to the sequence of SEQ ID NO:4 provided herein. Examples of FGFR1c variants include the sequences of NP_{—}001167534, NP_{—}001167535, NP_{—}001167536, NP_{—}001167537, NP_{—}001167538, NP_{—}075594, NP_{—}075598.

The amino acid sequence of full length human FGFR1c is:

SEQ ID NO: 4
MWSWKCLLFWAVLVTATLCTARPSPTLPEQAQPWGAPVEVESFLVHPGDL
LQLRCRLRDDVQSINWLRDGVQLAESNRTRITGEEVEVQDSVPADSGLYA
CVTSSPSGSDTTYFSVNVSDALPSSEDDDDDDDSSSEEKETDNTKPNRMP
VAPYWTSPEKMEKKLHAVPAAKTVKFKCPSSGTPNPTLRWLKNGKEFKPD
HRIGGYKVRYATWSIIMDSVVPSDKGNYTCIVENEYGSINHTYQLDVVER
SPHRPILQAGLPANKTVALGSNVEFMCKVYSDPQPHIQWLKHIEVNGSKI
GPDNLPYVQILKTAGVNTTDKEMEVLHLRNVSFEDAGEYTCLAGNSIGLS
HHSAWLTVLEALEERPAVMTSPLYLEIIIYCTGAFLISCMVGSVIVYKMK
SGTKKSDFHSQMAVHKLAKSIPLRRQVTVSADSSASMNSGVLLVRPSRLS
SSGTPMLAGVSEYELPEDPRWELPRDRLVLGKPLGEGCFGQVVLAEAIGL
DKDKPNRVTKVAVKMLKSDATEKDLSDLISEMEMMKMIGKHKNIINLLGA
CTQDGPLYVIVEYASKGNLREYLQARRPPGLEYCYNPSHNPEEQLSSKDL
VSCAYQVARGMEYLASKKCIHRDLAARNVLVTEDNVMKIADFGLARDIHH
IDYYKKTTNGRLPVKWMAPEALFDRIYTHQSDVWSFGVLLWEIFTLGGSP
YPGVPVEELFKLLKEGHRMDKPSNCTNELYMMMRDCWHAVPSQRPTFKQL
VEDLDRIVALTSNQEYLDLSMPLDQYSPSFPDTRSSTCSSGEDSVFSHEP
LPEEPCLPRHPAQLANGGLKRR,
(signal sequence underlined)

which is encoded by the DNA sequence

SEQ ID NO: 3
ATGTGGAGCTGGAAGTGCCTCCTCTTCTGGGCTGTGCTGGTCACAGCCAC
ACTCTGCACCGCTAGGCCGTCCCCGACCTTGCCTGAACAAGCCCAGCCCT
GGGGAGCCCCTGTGGAAGTGGAGTCCTTCCTGGTCCACCCCGGTGACCTG
CTGCAGCTTCGCTGTCGGCTGCGGGACGATGTGCAGAGCATCAACTGGCT
GCGGGACGGGGTGCAGCTGGCGGAAAGCAACCGCACCCGCATCACAGGGG
AGGAGGTGGAGGTGCAGGACTCCGTGCCCGCAGACTCCGGCCTCTATGCT
TGCGTAACCAGCAGCCCCTCGGGCAGTGACACCACCTACTTCTCCGTCAA
TGTTTCAGATGCTCTCCCCTCCTCGGAGGATGATGATGATGATGATGACT
CCTCTTCAGAGGAGAAAGAAACAGATAACACCAAACCAAACCGTATGCCC
GTAGCTCCATATTGGACATCACCAGAAAAGATGGAAAAGAAATTGCATGC
AGTGCCGGCTGCCAAGACAGTGAAGTTCAAATGCCCTTCCAGTGGGACAC
CAAACCCAACACTGCGCTGGTTGAAAAATGGCAAAGAATTCAAACCTGAC
CACAGAATTGGAGGCTACAAGGTCCGTTATGCCACCTGGAGCATCATAAT
GGACTCTGTGGTGCCCTCTGACAAGGGCAACTACACCTGCATTGTGGAGA
ATGAGTACGGCAGCATCAACCACACATACCAGCTGGATGTCGTGGAGCGG
TCCCCTCACCGGCCCATCCTGCAAGCAGGGTTGCCCGCCAACAAAACAGT
GGCCCTGGGTAGCAACGTGGAGTTCATGTGTAAGGTGTACAGTGACCCGC
AGCCGCACATCCAGTGGCTAAAGCACATCGAGGTGAATGGGAGCAAGATT
GGCCCAGACAACCTGCCTTATGTCCAGATCTTGAAGACTGCTGGAGTTAA
TACCACCGACAAAGAGATGGAGGTGCTTCACTTAAGAAATGTCTCCTTTG
AGGACGCAGGGGAGTATACGTGCTTGGCGGGTAACTCTATCGGACTCTCC
CATCACTCTGCATGGTTGACCGTTCTGGAAGCCCTGGAAGAGAGGCCGGC
AGTGATGACCTCGCCCCTGTACCTGGAGATCATCATCTATTGCACAGGGG
CCTTCCTCATCTCCTGCATGGTGGGGTCGGTCATCGTCTACAAGATGAAG
AGTGGTACCAAGAAGAGTGACTTCCACAGCCAGATGGCTGTGCACAAGCT
GGCCAAGAGCATCCCTCTGCGCAGACAGGTAACAGTGTCTGCTGACTCCA
GTGCATCCATGAACTCTGGGGTTCTTCTGGTTCGGCCATCACGGCTCTCC
TCCAGTGGGACTCCCATGCTAGCAGGGGTCTCTGAGTATGAGCTTCCCGA
AGACCCTCGCTGGGAGCTGCCTCGGGACAGACTGGTCTTAGGCAAACCCC
TGGGAGAGGGCTGCTTTGGGCAGGTGGTGTTGGCAGAGGCTATCGGGCTG
GACAAGGACAAACCCAACCGTGTGACCAAAGTGGCTGTGAAGATGTTGAA
GTCGGACGCAACAGAGAAAGACTTGTCAGACCTGATCTCAGAAATGGAGA
TGATGAAGATGATCGGGAAGCATAAGAATATCATCAACCTGCTGGGGGCC
TGCACGCAGGATGGTCCCTTGTATGTCATCGTGGAGTATGCCTCCAAGGG
CAACCTGCGGGAGTACCTGCAGGCCCGGAGGCCCCCAGGGCTGGAATACT
GCTACAACCCCAGCCACAACCCAGAGGAGCAGCTCTCCTCCAAGGACCTG
GTGTCCTGCGCCTACCAGGTGGCCCGAGGCATGGAGTATCTGGCCTCCAA
GAAGTGCATACACCGAGACCTGGCAGCCAGGAATGTCCTGGTGACAGAGG
ACAATGTGATGAAGATAGCAGACTTTGGCCTCGCACGGGACATTCACCAC
ATCGACTACTATAAAAAGACAACCAACGGCCGACTGCCTGTGAAGTGGAT
GGCACCCGAGGCATTATTTGACCGGATCTACACCCACCAGAGTGATGTGT
GGTCTTTCGGGGTGCTCCTGTGGGAGATCTTCACTCTGGGCGGCTCCCCA
TACCCCGGTGTGCCTGTGGAGGAACTTTTCAAGCTGCTGAAGGAGGGTCA
CCGCATGGACAAGCCCAGTAACTGCACCAACGAGCTGTACATGATGATGC
GGGACTGCTGGCATGCAGTGCCCTCACAGAGACCCACCTTCAAGCAGCTG
GTGGAAGACCTGGACCGCATCGTGGCCTTGACCTCCAACCAGGAGTACCT
GGACCTGTCCATGCCCCTGGACCAGTACTCCCCCAGCTTTCCCGACACCC
GGAGCTCTACGTGCTCCTCAGGGGAGGATTCCGTCTTCTCTCATGAGCCG
CTGCCCGAGGAGCCCTGCCTGCCCCGACACCCAGCCCAGCTTGCCAATGG
CGGACTCAAACGCCGC,
(signal sequence underlined).

As stated herein, the term “FGFR1c polypeptide” refers to a FGFR1c polypeptide comprising the human amino acid sequences SEQ ID NO:4. The term “FGFR1c polypeptide,” however, also encompasses polypeptides comprising an amino acid sequence that differs from the amino acid sequence of a naturally occurring FGFR1c polypeptide sequence, e.g., SEQ ID NO:4, by one or more amino acids, such that the sequence is at least 85% identical to SEQ ID NO:4. FGFR1c polypeptides can be generated by introducing one or more amino acid substitutions or a fragment of the receptor, either conservative or non-conservative and using naturally or non-naturally occurring amino acids, at particular positions of the FGFR1c polypeptide.

Nucleic acid sequences encoding a FGFR1c polypeptide provided herein, including those degenerate to SEQ ID NO:3, and those encoding polypeptide variants of SEQ ID NO:4 form other aspects of the instant disclosure.

### II.C. FGFR1b Polypeptide and Polynucleotide Sequences

In vivo, FGFR1b is expressed as a contiguous amino acid sequence comprising a signal sequence. Variants of FGFR1b are known and form aspects of the term “FGFR1b.” The amino acid sequence of full length human FGFR1b is:

SEQ ID NO: 6
MWSWKCLLFWAVLVTATLCTARPSPTLPEQAQPWGAPVEVESFLVHPGDL
LQLRCRLRDDVQSINWLRDGVQLAESNRTRITGEEVEVQDSVPADSGLYA
CVTSSPSGSDTTYFSVNVSDALPSSEDDDDDDDSSSEEKETDNTKPNRMP
VAPYWTSPEKMEKKLHAVPAAKTVKFKCPSSGTPNPTLRWLKNGKEFKPD
HRIGGYKVRYATWSIIMDSVVPSDKGNYTCIVENEYGSINHTYQLDVVER
SPHRPILQAGLPANKTVALGSNVEFMCKVYSDPQPHIQWLKHIEVNGSKI
GPDNLPYVQILKHSGINSSDAEVLTLFNVTEAQSGEYVCKVSNYIGEANQ
SAWLTVTRPVAKALEERPAVMTSPLYLEIIIYCTGAFLISCMVGSVIVYK
MKSGTKKSDFHSQMAVHKLAKSIPLRRQVTVSADSSASMNSGVLLVRPSR
LSSSGTPMLAGVSEYELPEDPRWELPRDRLVLGKPLGEGCFGQVVLAEAI
GLDKDKPNRVTKVAVKMLKSDATEKDLSDLISEMEMMKMIGKHKNIINLL
GACTQDGPLYVIVEYASKGNLREYLQARRPPGLEYCYNPSHNPEEQLSSK
DLVSCAYQVARGMEYLASKKCIHRDLAARNVLVTEDNVMKIADFGLARDI
HHIDYYKKTTNGRLPVKWMAPEALFDRIYTHQSDVWSFGVLLWEIFTLGG
SPYPGVPVEELFKLLKEGHRMDKPSNCTNELYMMMRDCWHAVPSQRPTFK
QLVEDLDRIVALTSNQEYLDLSMPLDQYSPSFPDTRSSTCSSGEDSVFSH
EPLPEEPCLPRHPAQLANGGLKRR,
(signal sequence underlined)

which is encoded by the DNA sequence

SEQ ID NO: 5
ATGTGGAGCTGGAAGTGCCTCCTCTTCTGGGCTGTGCTGGTCACAGCCAC
ACTCTGCACCGCTAGGCCGTCCCCGACCTTGCCTGAACAAGCCCAGCCCT
GGGGAGCCCCTGTGGAAGTGGAGTCCTTCCTGGTCCACCCCGGTGACCTG
CTGCAGCTTCGCTGTCGGCTGCGGGACGATGTGCAGAGCATCAACTGGCT
GCGGGACGGGGTGCAGCTGGCGGAAAGCAACCGCACCCGCATCACAGGGG
AGGAGGTGGAGGTGCAGGACTCCGTGCCCGCAGACTCCGGCCTCTATGCT
TGCGTAACCAGCAGCCCCTCGGGCAGTGACACCACCTACTTCTCCGTCAA
TGTTTCAGATGCTCTCCCCTCCTCGGAGGATGATGATGATGATGATGACT
CCTCTTCAGAGGAGAAAGAAACAGATAACACCAAACCAAACCGTATGCCC
GTAGCTCCATATTGGACATCACCAGAAAAGATGGAAAAGAAATTGCATGC
AGTGCCGGCTGCCAAGACAGTGAAGTTCAAATGCCCTTCCAGTGGGACAC
CAAACCCAACACTGCGCTGGTTGAAAAATGGCAAAGAATTCAAACCTGAC
CACAGAATTGGAGGCTACAAGGTCCGTTATGCCACCTGGAGCATCATAAT
GGACTCTGTGGTGCCCTCTGACAAGGGCAACTACACCTGCATTGTGGAGA
ATGAGTACGGCAGCATCAACCACACATACCAGCTGGATGTCGTGGAGCGG
TCCCCTCACCGGCCCATCCTGCAAGCAGGGTTGCCCGCCAACAAAACAGT
GGCCCTGGGTAGCAACGTGGAGTTCATGTGTAAGGTGTACAGTGACCCGC
AGCCGCACATCCAGTGGCTAAAGCACATCGAGGTGAATGGGAGCAAGATT
GGCCCAGACAACCTGCCTTATGTCCAGATCTTGAAGCATTCGGGGATTAA
TAGCTCGGATGCGGAGGTGCTGACCCTGTTCAATGTGACAGAGGCCCAGA
GCGGGGAGTATGTGTGTAAGGTTTCCAATTATATTGGTGAAGCTAACCAG
TCTGCGTGGCTCACTGTCACCAGACCTGTGGCAAAAGCCCTGGAAGAGAG
GCCGGCAGTGATGACCTCGCCCCTGTACCTGGAGATCATCATCTATTGCA
CAGGGGCCTTCCTCATCTCCTGCATGGTGGGGTCGGTCATCGTCTACAAG
ATGAAGAGTGGTACCAAGAAGAGTGACTTCCACAGCCAGATGGCTGTGCA
CAAGCTGGCCAAGAGCATCCCTCTGCGCAGACAGGTAACAGTGTCTGCTG
ACTCCAGTGCATCCATGAACTCTGGGGTTCTTCTGGTTCGGCCATCACGG
CTCTCCTCCAGTGGGACTCCCATGCTAGCAGGGGTCTCTGAGTATGAGCT
TCCCGAAGACCCTCGCTGGGAGCTGCCTCGGGACAGACTGGTCTTAGGCA
AACCCCTGGGAGAGGGCTGCTTTGGGCAGGTGGTGTTGGCAGAGGCTATC
GGGCTGGACAAGGACAAACCCAACCGTGTGACCAAAGTGGCTGTGAAGAT
GTTGAAGTCGGACGCAACAGAGAAAGACTTGTCAGACCTGATCTCAGAAA
TGGAGATGATGAAGATGATCGGGAAGCATAAGAATATCATCAACCTGCTG
GGGGCCTGCACGCAGGATGGTCCCTTGTATGTCATCGTGGAGTATGCCTC
CAAGGGCAACCTGCGGGAGTACCTGCAGGCCCGGAGGCCCCCAGGGCTGG
AATACTGCTACAACCCCAGCCACAACCCAGAGGAGCAGCTCTCCTCCAAG
GACCTGGTGTCCTGCGCCTACCAGGTGGCCCGAGGCATGGAGTATCTGGC
CTCCAAGAAGTGCATACACCGAGACCTGGCAGCCAGGAATGTCCTGGTGA
CAGAGGACAATGTGATGAAGATAGCAGACTTTGGCCTCGCACGGGACATT
CACCACATCGACTACTATAAAAAGACAACCAACGGCCGACTGCCTGTGAA
GTGGATGGCACCCGAGGCATTATTTGACCGGATCTACACCCACCAGAGTG
ATGTGTGGTCTTTCGGGGTGCTCCTGTGGGAGATCTTCACTCTGGGCGGC
TCCCCATACCCCGGTGTGCCTGTGGAGGAACTTTTCAAGCTGCTGAAGGA
GGGTCACCGCATGGACAAGCCCAGTAACTGCACCAACGAGCTGTACATGA
TGATGCGGGACTGCTGGCATGCAGTGCCCTCACAGAGACCCACCTTCAAG
CAGCTGGTGGAAGACCTGGACCGCATCGTGGCCTTGACCTCCAACCAGGA
GTACCTGGACCTGTCCATGCCCCTGGACCAGTACTCCCCCAGCTTTCCCG
ACACCCGGAGCTCTACGTGCTCCTCAGGGGAGGATTCCGTCTTCTCTCAT
GAGCCGCTGCCCGAGGAGCCCTGCCTGCCCCGACACCCAGCCCAGCTTGC
CAATGGCGGACTCAAACGCCGC,
(signal sequence underlined).

As stated herein, the term “FGFR1b polypeptide” refers to a FGFR1b polypeptide comprising the human amino acid sequences SEQ ID NO:6. The term “FGFR1b polypeptide,” however, also encompasses polypeptides comprising an amino acid sequence that differs from the amino acid sequence of a naturally occurring FGFR1b polypeptide sequence, e.g., SEQ ID NO:6, by one or more amino acids, such that the sequence is at least 85% identical to SEQ ID NO:6. FGFR1b polypeptides can be generated by introducing one or more amino acid substitutions or a fragment of the receptor, either conservative or non-conservative and using naturally or non-naturally occurring amino acids, at particular positions of the FGFR1b polypeptide.

Nucleic acid sequences encoding a FGFR1b polypeptide provided herein, including those degenerate to SEQ ID NO:5, and those encoding polypeptide variants of SEQ ID NO:6 form other aspects of the instant disclosure.

### II.D. Vectors for Expression of Recombinant Materials

In some embodiments, the provided method can be performed using an in vitro assay system. In such an assay system the components of the assay can be expressed recombinantly and transferred to a substrate (e.g., a welled plate, such as a 96 well plate) for performing the method. The relevant proteins can be expressed as follows.

In order to express the nucleic acid sequences provided herein (e.g., nucleic acids encoding FGFR1c and β-Klotho), the appropriate coding sequences, e.g., SEQ ID NOs:1 and 3, can be cloned into a suitable vector and after introduction in a suitable host, the sequence can be expressed to produce the encoded polypeptide according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). The invention also relates to such vectors comprising a nucleic acid sequence according to the invention.

A “vector” refers to a delivery vehicle that (a) promotes the expression of a polypeptide-encoding nucleic acid sequence; (b) promotes the production of the polypeptide therefrom; (c) promotes the transfection/transformation of target cells therewith; (d) promotes the replication of the nucleic acid sequence; (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid. A vector can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

A recombinant expression vector can be designed for expression of a recombinant protein in prokaryotic (e.g., E. coli) or eukaryotic cells (e.g., insect cells, using baculovirus expression vectors, yeast cells, or mammalian cells). Representative host cells include those hosts typically used for cloning and expression, including Escherichia coli strains TOP10F′, TOP10, DH10B, DH5a, HB101, W3110, BL21(DE3) and BL21 (DE3)pLysS, BLUESCRIPT (Stratagene), pIN vectors (Van Heeke & Schuster, J. Biol. Chem. 264: 5503-5509 (1989); pET vectors (Novagen, Madison, Wis.). Alternatively, the recombinant expression vector can be transcribed and translated in vitro, for example using T7 promoter regulatory sequences and T7 polymerase and an in vitro translation system. Preferably, the vector contains a promoter upstream of the cloning site containing the nucleic acid sequence encoding the polypeptide. Examples of promoters, which can be switched on and off, include the lac promoter, the T7 promoter, the trc promoter, the tac promoter and the trp promoter.

Thus, provided herein are vectors comprising a nucleic acid sequence encoding β-Klotho or FGFR1c that facilitate the expression of recombinant β-Klotho or FGFR1c. In various embodiments, the vectors comprise an operably linked nucleotide sequence which regulates the expression of β-Klotho or FGFR1c. A vector can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter), effective poly (A) termination sequences, an origin of replication for plasmid product in E. coli, an antibiotic resistance gene as a selectable marker, and/or a convenient cloning site (e.g., a polylinker). Vectors also can comprise an inducible promoter as opposed to a constitutive promoter such as CMV IE. In one aspect, a nucleic acid comprising a sequence encoding a β-Klotho or FGFR1c polypeptide which is operatively linked to a tissue specific promoter which promotes expression of the sequence in a metabolically-relevant tissue, such as liver or pancreatic tissue is provided.

### II.E. Host Cells for Expression of Recombinant Materials

In another aspect of the instant disclosure, host cells comprising the β-Klotho and FGFR1c nucleic acids and vectors disclosed herein are provided. In various embodiments, the vector or nucleic acid is integrated into the host cell genome, which in other embodiments the vector or nucleic acid is extra-chromosomal.

Recombinant cells, such as yeast, bacterial (e.g., E coli), and mammalian cells (e.g., immortalized mammalian cells) comprising such a nucleic acid, vector, or combinations of either or both thereof are provided. In various embodiments cells comprising a non-integrated nucleic acid, such as a plasmid, cosmid, phagemid, or linear expression element, which comprises a sequence coding for expression of a β-Klotho or FGFR1c polypeptide, are provided.

A vector comprising a nucleic acid sequence encoding a β-Klotho or FGFR1c polypeptide provided herein can be introduced into a host cell by transformation or by transfection. Methods of transforming a cell with an expression vector are well known.

A β-Klotho or FGFR1c-encoding nucleic acid can be positioned in and/or delivered to a host cell or host animal via a viral vector. Any suitable viral vector can be used in this capacity. A viral vector can comprise any number of viral polynucleotides, alone or in combination with one or more viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), or an intact virus particle comprising viral nucleic acids and a β-Klotho or FGFR1c polypeptide-encoding nucleic acid. A viral particle viral vector can comprise a wild-type viral particle or a modified viral particle. The viral vector can be a vector which requires the presence of another vector or wild-type virus for replication and/or expression (e.g., a viral vector can be a helper-dependent virus), such as an adenoviral vector amplicon. Typically, such viral vectors consist of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors.

### II.F. Isolation of a Recombinant β-Klotho or FGFR1 Polypeptides

A β-Klotho or FGFR1 polypeptide (e.g., FGFR1c) expressed as described herein can be isolated using standard protein purification methods. A β-Klotho or FGFR1c polypeptide can be isolated from a cell in which is it naturally expressed or it can be isolated from a cell that has been engineered to express β-Klotho or FGFR1c, for example a cell that does not naturally express β-Klotho or FGFR1c.

Standard protein purification methodology can be employed to isolate a β-Klotho or FGFR1c polypeptide, as well as associated materials and reagents, and are known in the art. See, e.g., The Tools of Biochemistry, Terrance G. Cooper, Wiley-Interscience (1977); Handbook of Process Chromatography: A Guide to Optimization, Scale-up and Validation, Gail Sofer and Lars Hagel, Academic Press (1997). Exemplary methods of purifying a β-Klotho or FGFR1c polypeptide are also provided in the Examples herein below.

II.G. Isolation of Membranes Comprising β-Klotho and/or FGFR1 Polypeptides

β-Klotho and FGFR1 are expressed in vivo as membrane-bound polypeptides. Accordingly, when the disclosed methods are performed in an in vitro embodiment, these components of the signaling assay can be in the form of isolated cell membranes expressing the proteins. This embodiment of the method can be advantageous in that it does not require isolation of the β-Klotho and FGFR1 polypeptides to a pure form; instead, membranes expressing the proteins can be isolated from cells.

Membranes expressing β-Klotho and FGFR1 can be extracted from cells that express these proteins naturally or recombinantly. Methods for harvesting membranes are known and can be employed in the method. See, e.g., Nikaido, (1994) Methods Enzymol. 235:225-34; Membrane Protein Purification and Crystallization, Second Edition: A Practical Guide, 2nd ed, Hunte et al, eds, Academic Press; (2003); The Tools of Biochemistry, Terrance G. Cooper, Wiley-Interscience (1977).

Following isolation of membranes expressing the proteins, the membranes can be transferred to a substrate, such as a welled plate, and the method can be performed on that substrate.

### III. METHOD OF IDENTIFYING MODULATORS OF THE INTERACTION OF β-KLOTHO WITH FGFR1

In one aspect the disclosed method provides an approach to assess the ability of a test molecule to modulate the interaction of β-Klotho with FGFR1. This can lead to the identification of molecules that exhibit equivalent or enhanced activity compared to molecules that normally signal through the β-Klotho/FGFR1-mediated signaling pathway. Examples of molecules that signal through this pathway include FGF21 and FGF19, among others. Thus, in one embodiment the disclosed method can be used as a screen to identify molecules that derive biological activity by signaling through a complex comprising β-Klotho and FGFR1. A non-inclusive list of examples of the types of molecules that can be screened using the disclosed method include mutant forms of FGF21, mutant forms of FGF19, FGF21 analogs, including antibodies, peptibodies, Avimers™ and various other modalities that activate signaling through the b-Klotho/FGFR1-mediated signaling pathway, and FGF19 analogs, including antibodies, peptibodies, Avimers™ and various other modalities that activate signaling through the b-Klotho/FGFR1-mediated signaling pathway. In one application, the method can be used to identify potentially therapeutic molecules designed to activate the FGF21- or FGF19-mediated pathways and subsequently provide biological activities similar to those mediated by FGF19 and/or FGF21 in vivo.

The results of the assay system can be extrapolated to serve as an indicator of the degree to which a test molecule will affect FGF-mediated signaling and ultimately FGF-mediated activities at the systemic level. For example, a test molecule that provides a higher level of signaling in the assay system relative to the activity of a selected standard (e.g., an FGF such as FGF21 or FGF19) can be expected to provide a higher level of activity at the tissue level. In one example, a test molecule that provides a higher level of signaling in the assay system than FGF21 would be expected to provide an enhancement to the activity of FGF21 on a tissue level, such as an enhanced ability to lower blood glucose levels, blood insulin levels, circulating triglyceride levels and/or circulating cholesterol levels. In another example, a test molecule that provides a higher level of signaling in the assay system than FGF19 would be expected to provide an enhancement to the activity of FGF19 on a tissue level, such as an enhanced ability to lower blood glucose levels, blood insulin levels, circulating triglyceride levels and/or circulating cholesterol levels.

Methods of identifying a compound that specifically modulates the interaction of FGFR1 and β-Klotho are provided. In one embodiment the method comprises determining a baseline level of FGFR1-mediated signaling in a signaling assay system comprising β-Klotho and FGFR1, wherein the FGFR1-mediated signal is one or more of Erk phosphorylation, FGFR1 phosphorylation and FRS2 phosphorylation.

The signaling assay system can be an in vitro system or an in vivo system. In one embodiment of an in vitro assay, the assay comprises a β-Klotho polypeptide and an FGFR1 polypeptide. The polypeptides can be produced recombinantly using the methods provided herein and known in the art. The assay can be performed on any suitable surface, such as a plastic or glass welled plate, such as a plastic 96-welled plate. In another embodiment the method can be performed in vitro using cell membranes on which β-Klotho and/or FGFR1 are expressed. When an in vivo signaling assay system is employed the assay system can be performed on an animal, such as a mammal, e.g., a rat, a mouse, or a non-human primate.

The signaling assay generates one or more detectable signals that serve as a measurable indicator of FGFR1-mediated signaling and depends on the presence of FGFR1 and β-Klotho. A suitable signal can be any measurable output of the assay system, and examples of detectable signals include Erk phosphorylation, FGFR1 phosphorylation and FRS2 (fibroblast growth factor substrate 2) phosphorylation.

Initially, a baseline signal is generated. The baseline signal level is determined in the presence of β-Klotho, FGFR1 and a reference molecule. The reference molecule can be any molecule known to generate a detectable signal in the presence of β-Klotho and FGFR1. Examples of reference molecules include FGF19 and FGF21. In one particular example a goal of the method can be to identify a mimetic of FGF21 and consequently FGFR1-mediated signaling in the presence of FGF21 will be most relevant. In another example, a goal of the method can be to identify a mimetic of FGF19 and consequently FGFR1-mediated signaling in the presence of FGF19 will be most relevant.

After acquiring a baseline signal in the presence of the ternary signaling complex (FGFR1, β-Klotho and the reference molecule) a test compound is contacted with the signaling assay system. In one embodiment the contacting can be performed by adding an aliquot of a solution comprising the test molecule to the substrate on which the assay system is disposed. A test molecule can be any molecule known or suspected of signaling through the FGFR1-mediated signaling pathway. As noted herein, the method can be used to identify a mimetic of FGF19 and/or FGF21. Accordingly, the test molecule can be a mimetic or analog of these growth factors.

Continuing, after contacting a test molecule with the signal assay a level of FGFR1-mediated signaling in the presence of the test compound is detected. The signal should be of the same type measured when determining the baseline signal (e.g., ERK phosphorylation, FGFR1 phosphorylation, FRS2 phosphorylation, etc). The contacting can be achieved using any convenient means, for example formulating the test molecule in a buffered solution and transferring an aliquot from a stock to the substrate on which the method is being performed.

Following acquisition of both a baseline and a test signal level the level of FGFR1-mediated signaling in the presence of the test compound is compared with the baseline level of FGFR1-mediated signaling; a difference between the two signaling levels indicates that the test compound modulates the interaction of FGFR1 and β-Klotho. The comparison can be made in a statistically significant manner or it can be made in order to simply provide a relative indicator of the degree to which a test molecule modulates signaling.

The method can be performed under any conditions a researcher may deem convenient or desirable. For example, the method can be performed under sterile or unsterile conditions, as a single screen or as a substep of a larger screening effort.

### IV. PHARMACEUTICAL COMPOSITIONS COMPRISING IDENTIFIED MODULATORS

Pharmaceutical compositions comprising a compound that is identified using the disclosed methods are provided. Such pharmaceutical compositions can comprise a therapeutically effective amount of a compound that specifically modulates the interaction of FGFR1 and β-Klotho identified using the provided methods in admixture with a pharmaceutically or physiologically acceptable formulation agent selected for suitability with the mode of administration. The term “pharmaceutically acceptable carrier” or “physiologically acceptable carrier” as used herein refers to one or more formulation agents suitable for accomplishing or enhancing the delivery of a compound that specifically modulates the interaction of FGFR1 and β-Klotho identified using the provided methods into the body of a human or non-human subject. The term includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In some cases it will be preferable to include isotonic agents, for example, sugars, polyalcohols such as mannitol, sorbitol, or sodium chloride in a pharmaceutical composition. Pharmaceutically acceptable substances such as wetting or minor amounts of auxiliary substances such as wetting or emulsifying agents, preservatives or buffers, which enhance the shelf life or effectiveness of the compound that specifically modulates the interaction of FGFR1 and β-Klotho identified using the provided methods can also act as, or form a component of, a carrier. Acceptable pharmaceutically acceptable carriers are preferably nontoxic to recipients at the dosages and concentrations employed.

A pharmaceutical composition can contain formulation agent(s) for modifying, maintaining, or preserving, for example, the pH, osmolarity, viscosity, clarity, color, isotonicity, odor, sterility, stability, rate of dissolution or release, adsorption, or penetration of the composition. Suitable formulation agents include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON: THE SCIENCE AND PRACTICE OF PHARMACY, 19th edition, (1995); Berge et al., J. Pharm. Sci., 6661), 1-19 (1977). Additional relevant principles, methods, and agents are described in, e.g., Lieberman et al., PHARMACEUTICAL DOSAGE FORMS-DISPERSE SYSTEMS (2nd ed., vol. 3, 1998); Ansel et al., PHARMACEUTICAL DOSAGE FORMS & DRUG DELIVERY SYSTEMS (7th ed. 2000); Martindale, THE EXTRA PHARMACOPEIA (31st edition), Remington's PHARMACEUTICAL SCIENCES (16th-20^{th} and subsequent editions); The Pharmacological Basis Of Therapeutics, Goodman and Gilman, Eds. (9th ed.—1996); Wilson and Gisvolds' TEXTBOOK OF ORGANIC MEDICINAL AND PHARMACEUTICAL CHEMISTRY, Delgado and Remers, Eds. (10th ed., 1998). Principles of formulating pharmaceutically acceptable compositions also are described in, e.g., Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), incorporated herein by reference for any purpose).

The optimal pharmaceutical composition will be determined by a skilled artisan depending upon, for example, the intended route of administration, delivery format, and desired dosage (see, e.g., Remington's PHARMACEUTICAL SCIENCES, supra). Such compositions can influence the physical state, stability, rate of in vivo release, and rate of in vivo clearance of an identified modulator.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the present invention, compositions can be prepared for storage by mixing the selected composition having the desired degree of purity with optional formulation agents (Remington's PHARMACEUTICAL SCIENCES, supra) in the form of a lyophilized cake or an aqueous solution. Furthermore, the compound that specifically modulates the interaction of FGFR1 and β-Klotho identified using the provided methods can be formulated as a lyophilizate using appropriate excipients such as sucrose.

Pharmaceutical compositions comprising a compound that specifically modulates the interaction of FGFR1 and β-Klotho identified using the provided methods can be selected for parenteral delivery. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention can be in the form of a pyrogen-free, parenterally acceptable, aqueous solution comprising the desired compound that specifically modulates the interaction of FGFR1 and β-Klotho identified using the provided methods in a pharmaceutically acceptable vehicle. A particularly suitable vehicle for parenteral injection is sterile distilled water in which a compound that specifically modulates the interaction of FGFR1 and β-Klotho identified using the provided methods is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a compound that specifically modulates the interaction of FGFR1 and β-Klotho identified using the provided methods can be formulated as a dry powder for inhalation. Inhalation solutions can also be formulated with a propellant for aerosol delivery. In yet another embodiment, solutions can be nebulized. Pulmonary administration is further described in International Publication No. WO 94/20069, which describes the pulmonary delivery of chemically modified proteins.

It is also contemplated that certain formulations can be administered orally. In one embodiment of the present invention, compounds that specifically modulate the interaction of FGFR1 and β-Klotho identified using the provided methods that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized Additional agents can be included to facilitate absorption of the compound that specifically modulates the interaction of FGFR1 and β-Klotho identified using the provided methods. Diluents, flavorings, low melting point waxes, vegetable oils, lubricants, suspending agents, tablet disintegrating agents, and binders can also be employed.

Another pharmaceutical composition can involve an effective quantity of a compound that specifically modulates the interaction of FGFR1 and β-Klotho identified using the provided methods in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations comprising compounds that specifically modulate the interaction of FGFR1 and β-Klotho identified using the provided methods in sustained- or controlled-delivery formulations. Techniques for formulating a variety of other sustained- or controlled-delivery means, such as liposome carriers, bio-erodible microparticles or porous beads and depot injections, are also known to those skilled in the art (see, e.g., International Publication No. WO 93/15722, which describes the controlled release of porous polymeric microparticles for the delivery of pharmaceutical compositions, and Wischke & Schwendeman, 2008, Int. J. Pharm. 364: 298-327, and Freiberg & Zhu, 2004, Int. J. Pharm. 282: 1-18, which discuss microsphere/microparticle preparation and use). As described herein, a hydrogel is an example of a sustained- or controlled-delivery formulation.

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et al., 1983, Biopolymers 22: 547-56), poly(2-hydroxyethyl-methacrylate) (Langer et al., 1981, J. Biomed. Mater. Res. 15: 167-277 and Langer, 1982, Chem. Tech. 12: 98-105), ethylene vinyl acetate (Langer et al., supra) or poly-D(−)-3-hydroxybutyric acid (European Patent No. 0 133 988). Sustained-release compositions can also include liposomes, which can be prepared by any of several methods known in the art. See, e.g., Epstein et al., 1985, Proc. Natl. Acad. Sci. U.S.A. 82: 3688-92; and European Patent Nos. 0 036 676, 0 088 046, and 0 143 949.

A pharmaceutical composition comprising a molecule that specifically modulates the interaction of FGFR1 and β-Klotho identified using the provided methods to be used for in vivo administration typically should be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the present invention is directed to kits for producing a single-dose administration unit. The kits can each contain both a first container having a dried protein and a second container having an aqueous formulation. Also included within the scope of this invention are kits containing single and multi-chambered pre-filled syringes (e.g., liquid syringes and lyosyringes).

The effective amount of a pharmaceutical composition provided herein to be employed therapeutically will depend, for example, upon the therapeutic context and objectives. One skilled in the art will appreciate that the appropriate dosage levels for treatment will thus vary depending, in part, upon the molecule delivered, the indication for which a compound that specifically modulates the interaction of FGFR1 and β-Klotho identified using the provided methods is being used, the route of administration, and the size (body weight, body surface, or organ size) and condition (the age and general health) of the patient. Accordingly, the clinician can titer the dosage and modify the route of administration to obtain the optimal therapeutic effect. A typical dosage can range from about 0.1 μg/kg to up to about 100 mg/kg or more, depending on the factors mentioned above. In other embodiments, the dosage can range from 0.1 μg/kg up to about 100 mg/kg; or 1 μg/kg up to about 100 mg/kg; or 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, up to about 100 mg/kg. In yet other embodiments, the dosage can be 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1000 μg/kg, 2000 μg/kg, 3000 μg/kg, 4000 μg/kg, 5000 μg/kg, 6000 μg/kg, 7000 μg/kg, 8000 μg/kg, 9000 μg/kg or 10 mg/kg.

The frequency of dosing will depend upon the pharmacokinetic parameters of the molecule in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems (which may also be injected); or by implantation devices. Where desired, the compositions can be administered by bolus injection or continuously by infusion, or by implantation device.

Alternatively or additionally, the composition can be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

### EXAMPLES

The following examples, including the experiments conducted and results achieved, are provided for illustrative purposes only and are not to be construed as limiting the present invention.

### Example 1

### Signaling Assays

Various host cell lines (such as HEK293, CHO, L6 cells etc) were co-transfected with expression vectors for FGFR1c with or without β-Klotho. Following overnight serum starvation, cells were stimulated with vehicle or recombinant FGF19 or FGF21 for a short period of time such as 15 min and snap frozen in liquid nitrogen. Cell lysates were prepared for Western blot analysis using antibodies against phosphorylated FGF receptor (p-FGFR), phosphorylated FSR2 (p-FRS2), phosphorylated ERK1/2 (p-ERK) and total ERK1/2 (T-ERK). Antibodies were all purchased from Cell Signaling.

The described signaling assay can also be carried out in vivo. Liver and adipose tissues collected minutes to several hours after injection of recombinant FGF19 or FGF21 and their variants can be snap frozen in liquid nitrogen, homogenized in lysis buffer, and subjected to Western blot analysis using antibodies described above.

Signaling was also measured using a MSD assay. Cells in each well were lysed in 60 μl of complete lysis buffer and total and phosphorylated ERK were measured using an MSD whole cell lysate Phospho-ERK1/2 kit (Meso Scale Discovery) according to the manufacturer's instructions.

### Example 2

### Generation of FGFR1 Knockout Mice

FGFR1c knockout mice were generated by crossing mice with floxed FGFR1 mice and mice with an aP2 promoter driven Cre allele and backcrossed to yield relatively pure c57/B6 background under the control of fat specific aP2 promoter. The resulting mice do not express any of the FGFR1 isoforms (FGFR1b or FGFR1c).

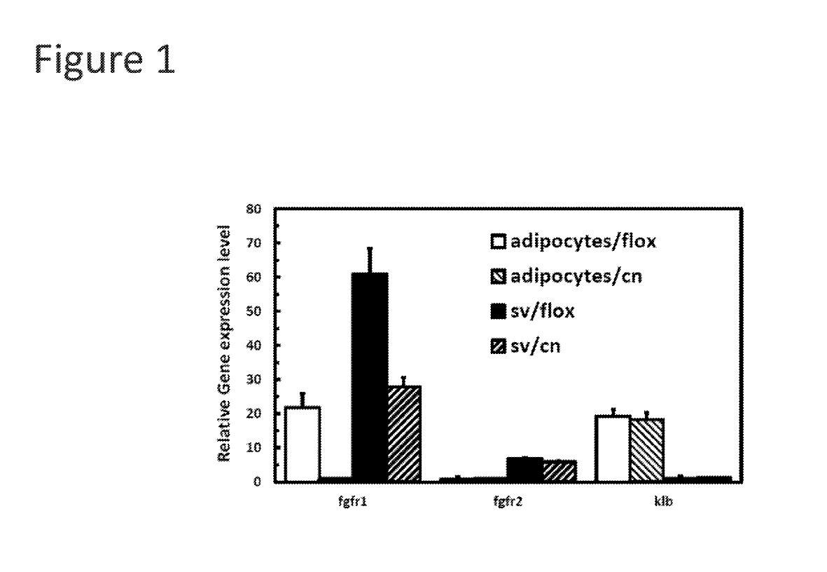

FIG. 1 shows the expression levels of FGFR1, FGFR2 and b-Klotho in adipocytes in the knockout mice.

### Example 3

### ERK Signaling, Body Weight and Glucose Metabolism in FGFR1KO Mice in which Obesity and Insulin Resistance was Induced

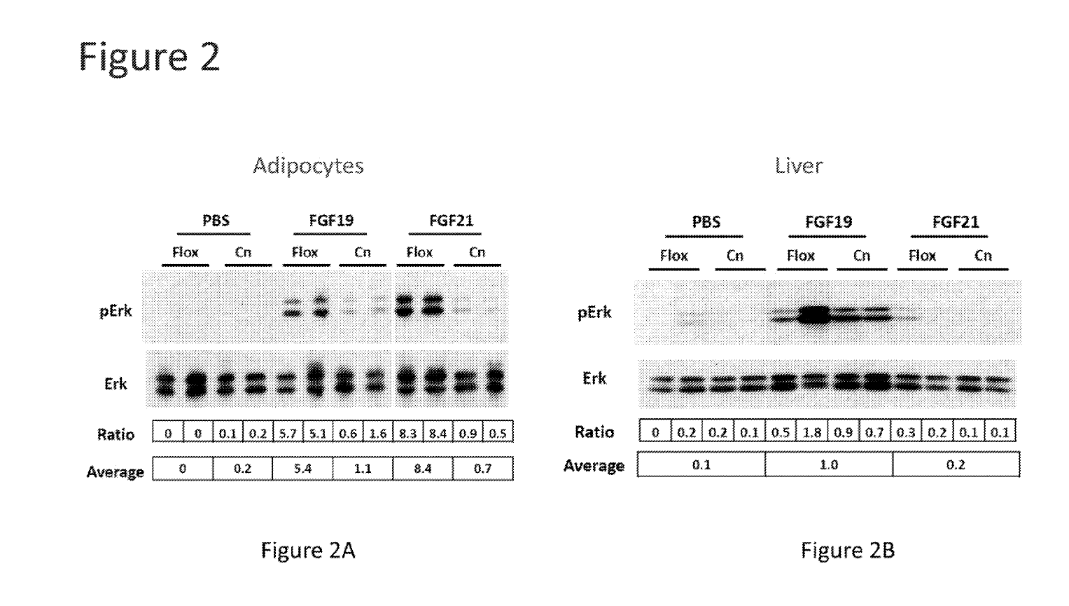

Wild type and FGFR1 KO mice were first put on high fat diet to induce obesity and insulin resistance (a “DIO” model). Both groups were then dosed daily IP with 5 mg/kg of FGF19 or FGF21 in PBS for two weeks and sacrificed on day 16. A graphic depiction of the study plan is shown in FIG. 2.

### Example 3A

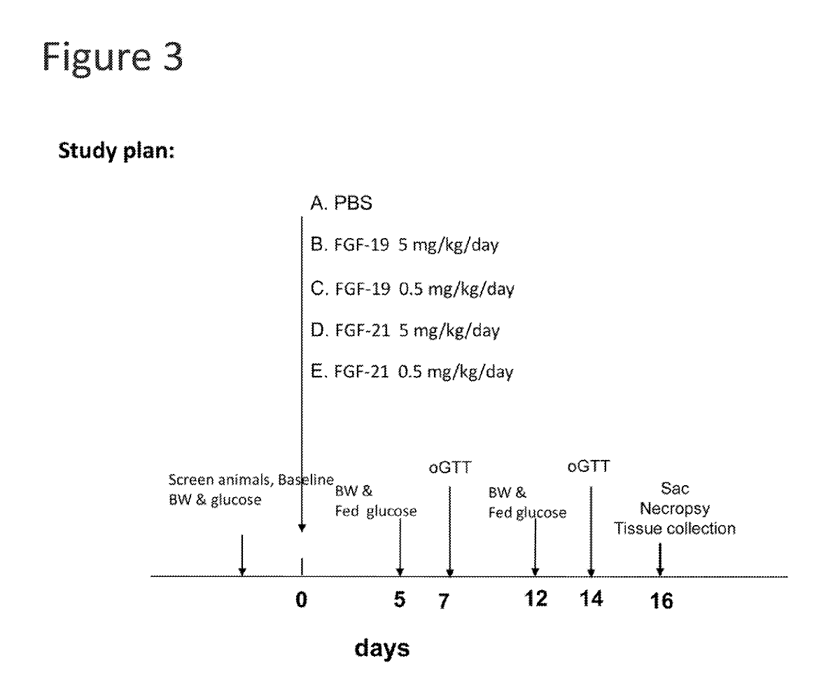

Tissues were harvested 20 min post injection followed by immunoblot analysis using Erk and pErk antibodies. Ratio of pErk/Erk was calculated from density of bands determined with ImagJ software. Results and average of two mice are indicated in FIG. 3 (Flox=control mice bearing loxP flanked FGFR1; Cn=mice bearing FGFR1-deficient adipocytes).

FIG. 3 (left panel) demonstrates that activation of Erk in adipocytes by FGF19 and FGF21 are mediated through FGFR1c. The fat specific FGFR1c KO completely abolished the ability of both FGF21 and FGF19 to induce signaling in adipocytes (FIG. 2, left panel).

FIG. 3 (right panel) demonstrates that in liver FGF19-induced signaling is still intact in these animals, suggesting that the defect in the FGFR signaling in fat is due to the specific KO of FGFR1 from adipocytes.

### Example 3B

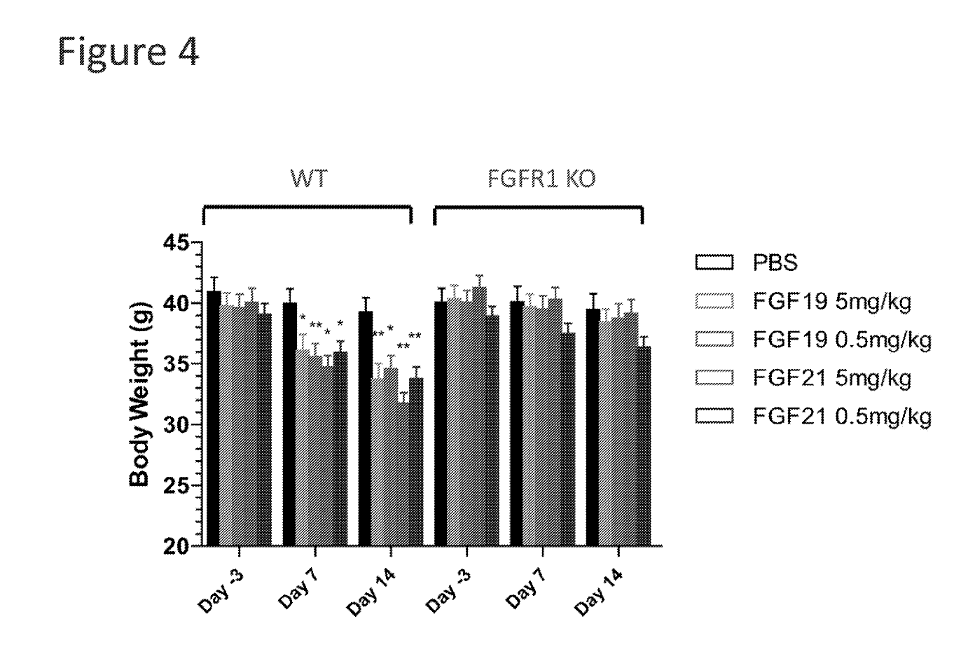

The effect of the FGFR1 knockout on induced body weight reduction was studied. As shown in FIG. 4, the DIO FGFR1c KO abolished FGF19 and FGF21 induced body weight reduction over the course of the 14 day study.

### Example 3C

The effect of the FGFR1 knockout on glucose levels was also studied. OGTTs were run on both groups of animals after a 1 week time period and after a 2 week time period.

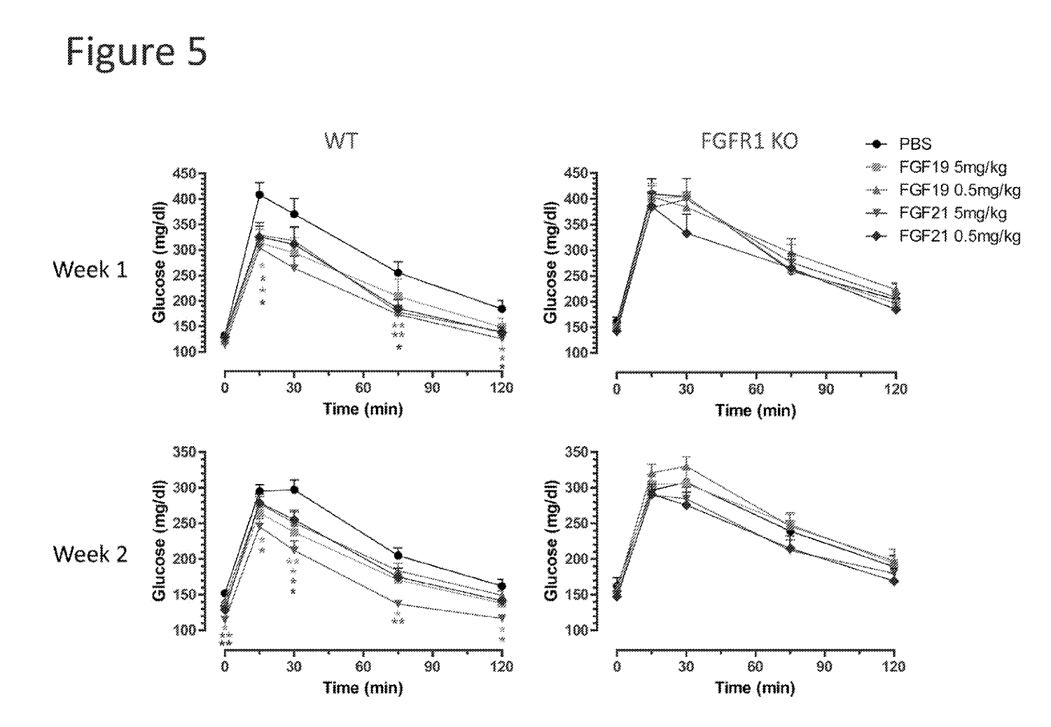

As shown in FIG. 5, the obese FGFR1c KO abolished FGF19- and FGF21-induced improvement on OGTT. The results were consistent after the one week (upper plots) and two week (lower plots) time periods.

### Example 3D

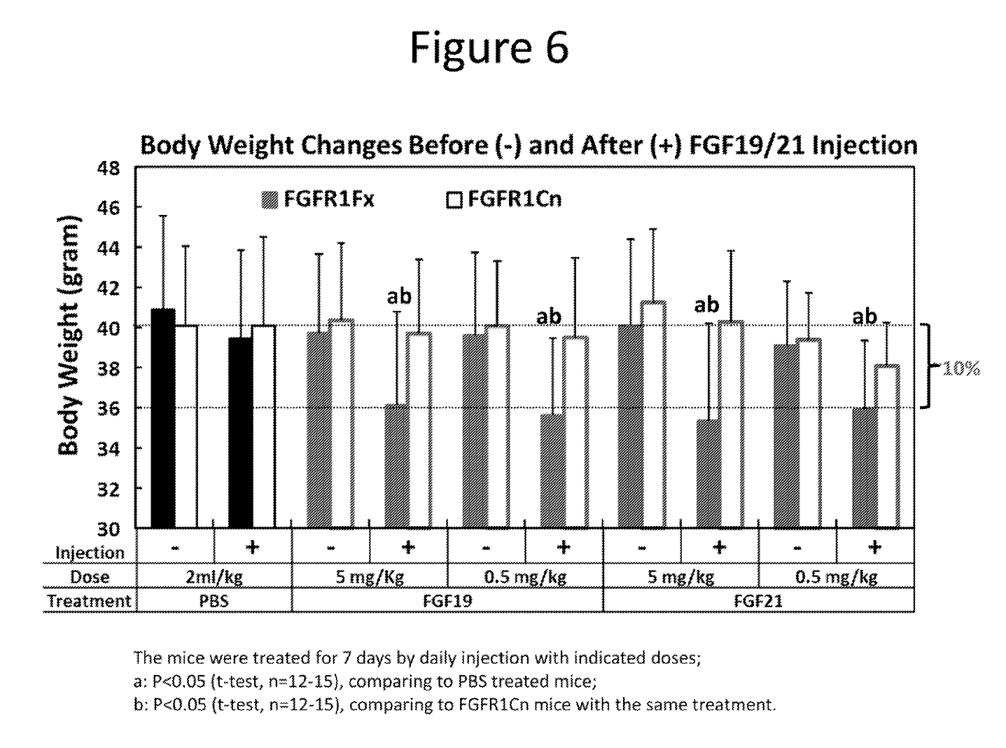

As shown in FIG. 6 the FGFR1 knockout also abolished the ability of both FGF21 and FGF19 to reduce body weight following daily IP injection of the doses of FGF21 and FGF19 over a 14 day period, as indicated in FIG. 6.

### Example 3E

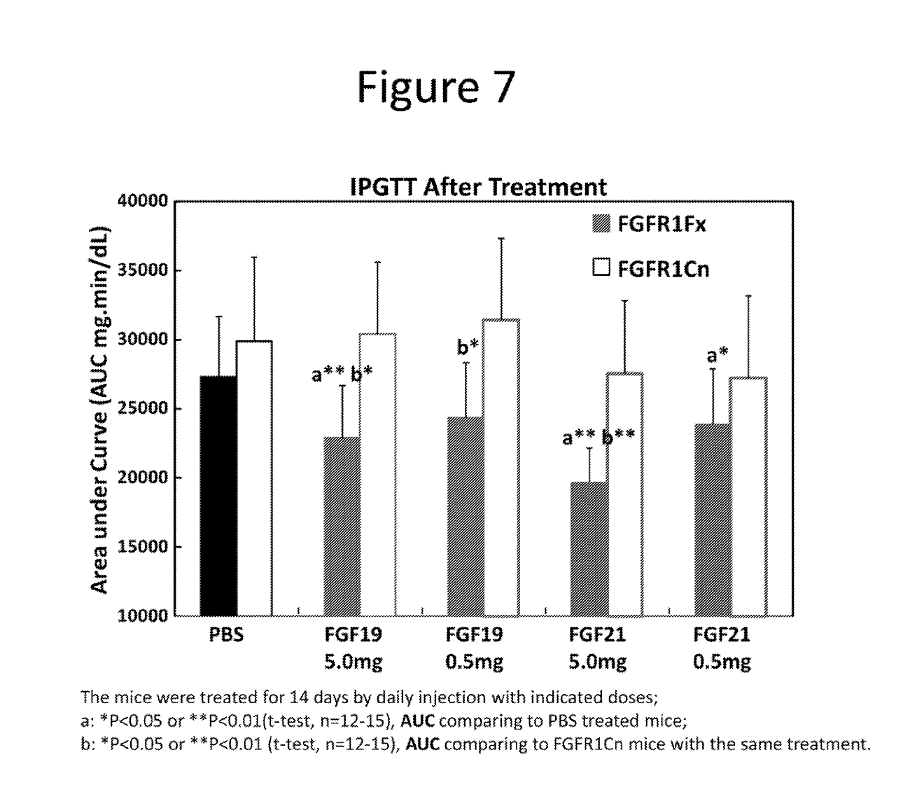

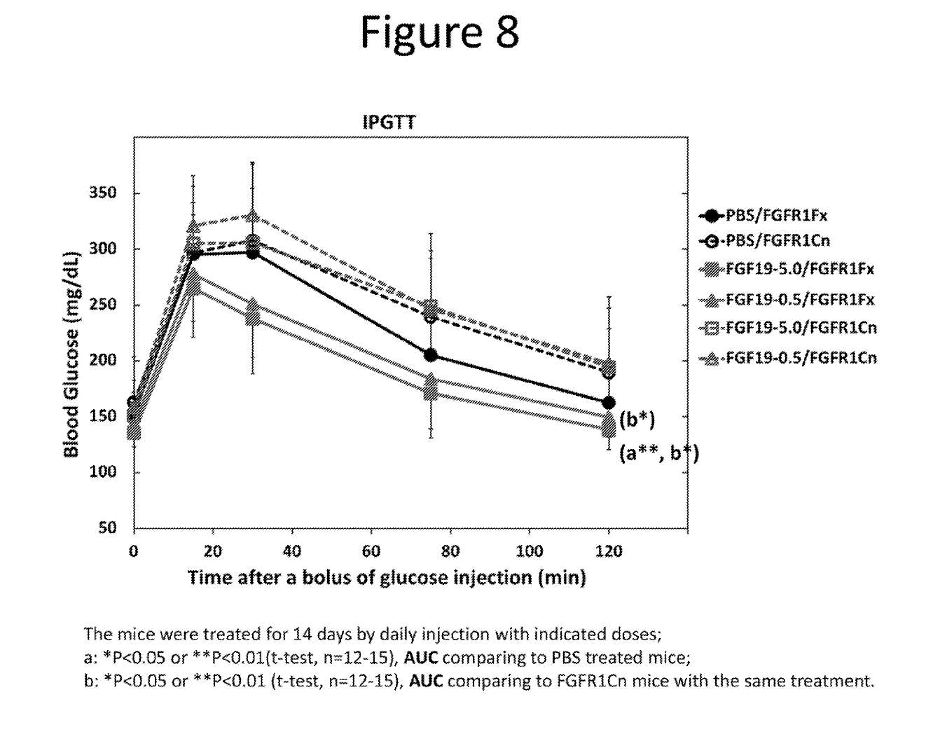

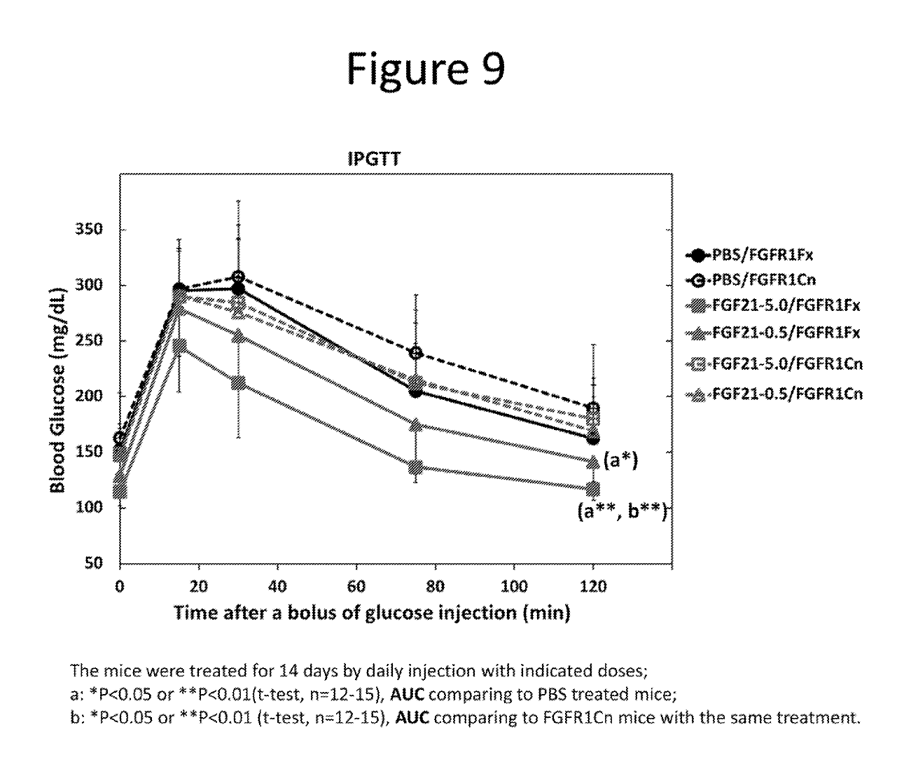

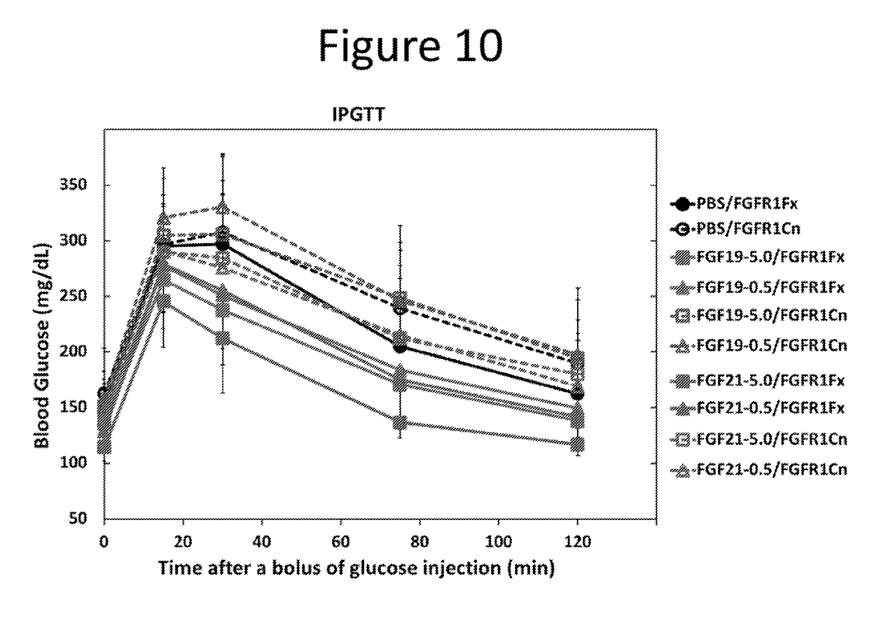

FIG. 7 demonstrates that the FGFR1 knockout also abolished the ability of FGF21 and FGF19 to improve glucose metabolism following 14 days of daily IP injections of the doses of FGF21 and FGF19 indicated in FIG. 7 over a 14 day period. FIGS. 8 and 9 show blood glucose levels in the animals following an OGGT in animals treated with FGF19 (FIG. 8) and FGF21 (FIG. 9). FIG. 10 summarizes the data of FIGS. 8 and 9.

Collectively, this data suggests a central role of fat as the target tissue and FGFR1c/β-Klotho as the essential receptor mediating the beneficial metabolic effects of FGF21 and FGF19.

### Example 4

### Receptor-Ligand Interaction Studies

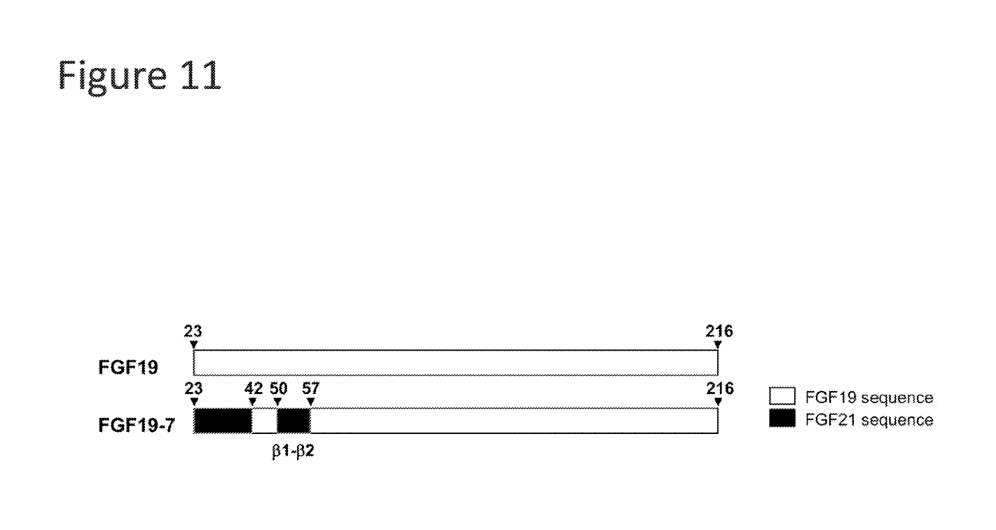

In order to further study the specific regions of FGF19 that are involved in the FGFR1-mediated signaling suggested by the animal studies described in Examples 1-3, the chimeric protein shown in FIG. 11 was generated and was termed FGF19-7. FGF19-7 comprises a FGF19 scaffold into which corresponding residues from FGF21 were swapped. More specifically, residues 23 to 42 of full length FGF19 (namely residues RPLAFSDAGPHVHYGWGDPI (SEQ ID NO:21) of SEQ ID NO:10) were replaced with residues 29 to 44 of full length FGF21, (namely residues HPIPDSSPLLQFGGQV (SEQ ID NO:7 of SEQ ID NO:16) and residues 50-57 of FGF19, corresponding to the 131-132 loop (namely residues SGPHGLSS (SEQ ID NO:22) of SEQ ID NO:10), were replace with residues 51 to 57 of FGF21 of full length FGF21 (namely residues DDAQQTE (SEQ ID NO:8) of SEQ ID NO:10). The amino acid and coding sequences of FGF19-7 are shown in SEQ ID NOs:24 and 23 respectively.

For expression of recombinant proteins, wild type FGF19 (residues 23-216, without secretory leader peptide, SEQ ID NO:12), FGF21 (residues 29-209, without secretory leader peptide, SEQ ID NO:18) and construct 19-7 were cloned into the pET30 vectors (Novagen). DNA constructs were transformed into BL21(DE3) E. coli (Novagen). Protein expression was induced with IPTG at 37° C. The purification process was the same as previously described (Wu et al., (2008) J. Biol. Chem. 283(48):33304-9). FGF21 (residues 29-209 of full length FGF21, i.e., the mature form of FGF21 without the secretory leader peptide, SEQ ID NO:18) was purified as previously described (Xu et al., (2009) Diabetes 58(1):250-9).

### Example 4A

### Effect of FGF19, FGF21 and FGF19-7 on Signaling

FGF19-7 was studied alongside FGF21 and wild-type FGF19 in an L6 cells expressing (3-Klotho and an FGFR, namely FGFR1c, FGFR2c, FGFR3c or FGFR4. Phosphorylation of ERK was used as a gauge of signaling.

Briefly, L6 cells were maintained in Dulbecco's modified Eagle's medium supplemented with 10% fetal bovine serum and penicillin/streptomycin. Cells were transfected with expression vectors using the Lipofectamine 2000 transfection reagent (Invitrogen) according to the manufacturer's protocol.

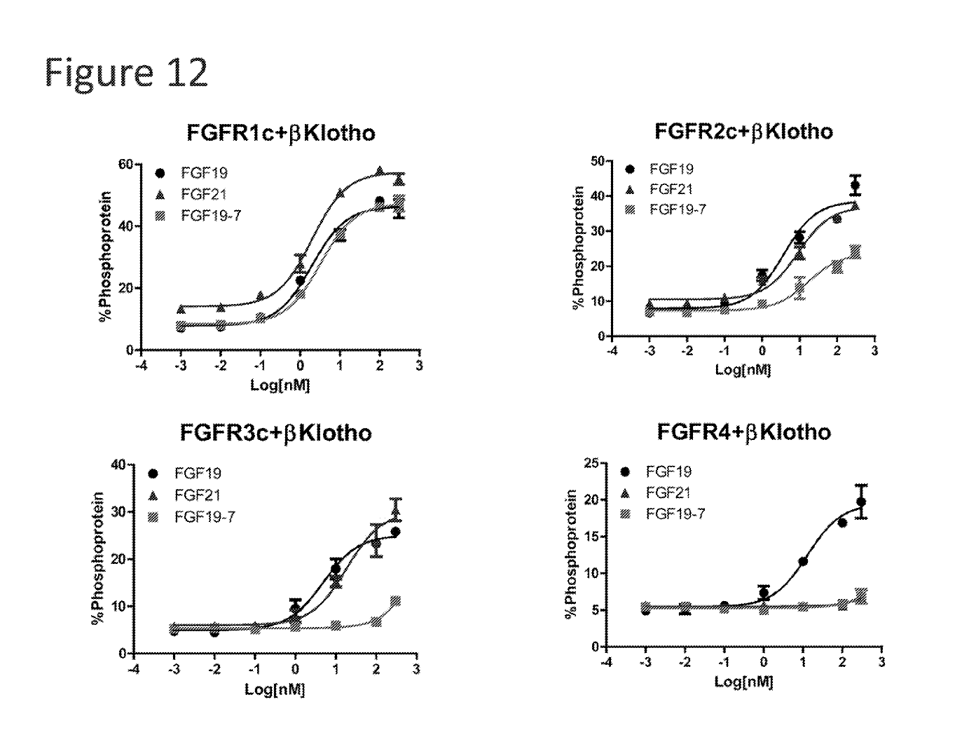

Signaling in response to FGF treatment was then assessed by measuring phospho-ERK (p-ERK) levels by a semiquantitative MSD assay format. While FGF19 was able to induce ERK phosphorylation with all four FGFRs: 1c, 2c, 3c and 4 co-transfected with β-Klotho in L6 cells, FGF21 activated only FGFRs 1c, 2c, and 3c with β-Klotho but not FGFR4 (FIG. 12). However, the receptor specificity profile of FGF 19-7 is significantly different from both FGF 19 and FGF21. While FGF19-7 fully activated FGFR1c/β-Klotho, it only partially activated FGFR2c/β-Klotho and no significant activation was observed on either FGFR3c or FGFR4 in the presence of β-Klotho, therefore, FGF19-7 is now biased toward FGFR1c/β-Klotho receptor complex (FIG. 12).

### Example 4B

### In Vivo Studies of FGF 19 and FGF 19-7

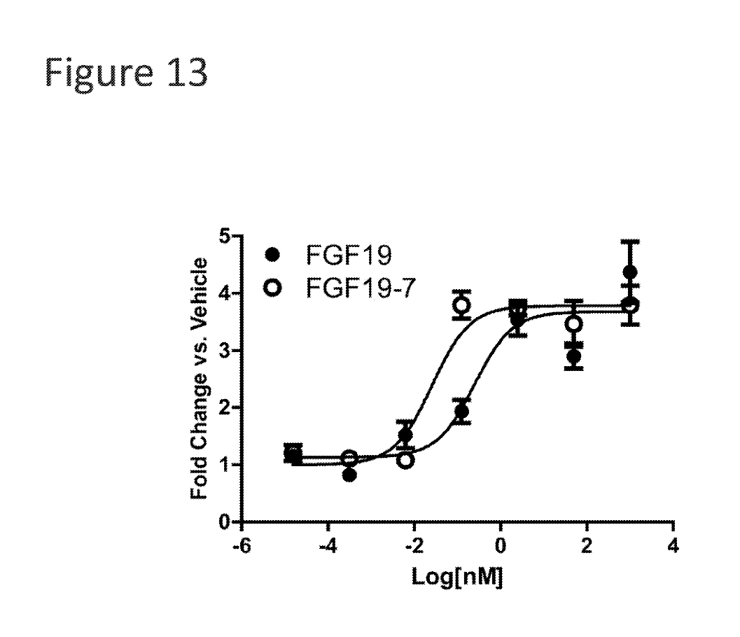

FGF 19 and FGF 19-7 were tested for their ability to influence a variety of metabolic parameters in vivo. DIO mice were used as a model system and the reagents were dosed by daily injection. The results of the in vivo study are presented in FIGS. 13-17.

### Two Week Study

FIG. 13 is a plot showing the effect of FGF19 and FGF19-7 on glucose uptake and highlights that FGF19-7 is comparable to FGF19 at increasing glucose uptake into adipocytes.

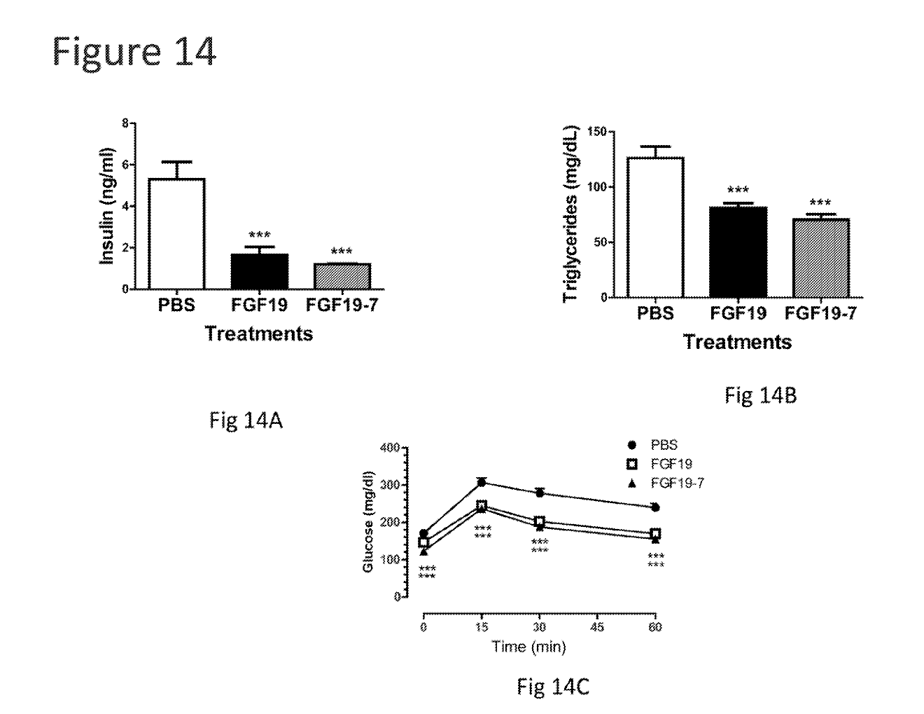

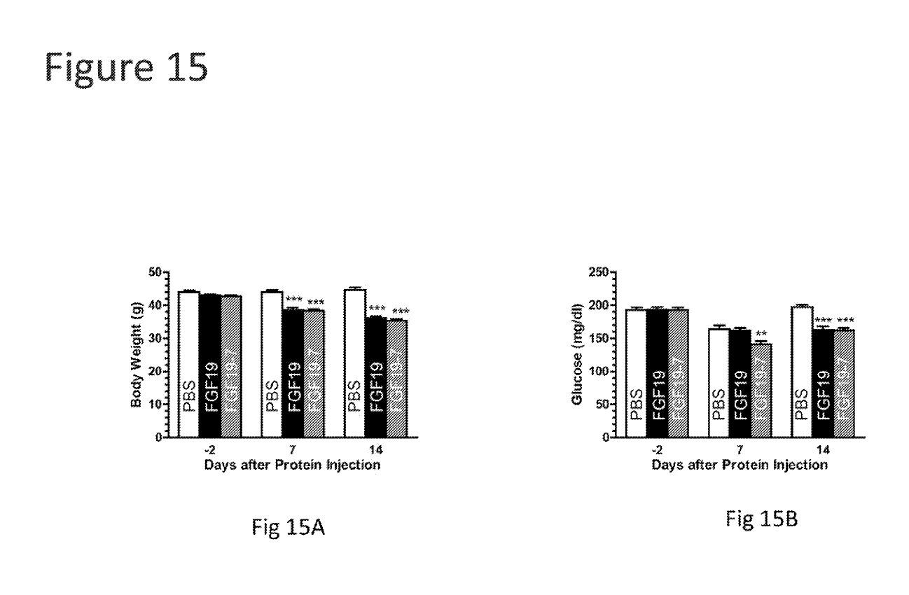

The ability of FGF19-7 to regulate glucose metabolism in vivo was demonstrated in both a diet-induced-obesity (DIO) model as well as the leptin deficient ob/ob mice. 14-weeks-old male B6D2F1 mice (fed on a high-fat diet for 8 weeks) were divided into 3 groups (n=12) based on body weight and glucose. Mice were then injected intraperitoneally (i.p.) with PBS, 1 mg/kg FGF19, or 1 mg/kg FGF19-7 daily for a period of 2 weeks. Compared to FGF19, FGF19-7 showed equally reduction in body weight throughout the study (FIG. 15A), equally reduction in plasma insulin (FIG. 14A) and triglycerides (FIG. 14B) levels. FGF19-7 group also showed a slightly better reduction in fasting glucose level where significant reduction was observed at day 7 post-start of the treatment where FGF 19 group was not yet significant. An oral glucose tolerance test was performed at the end of the 2-week treatment to assess the ability of the animals to dispose a glucose challenge. As shown in FIG. 14C, both FGF19-7 and FGF19 treatments significantly improved the response of animals to the oral glucose challenge (OGTT) to a similar extent.

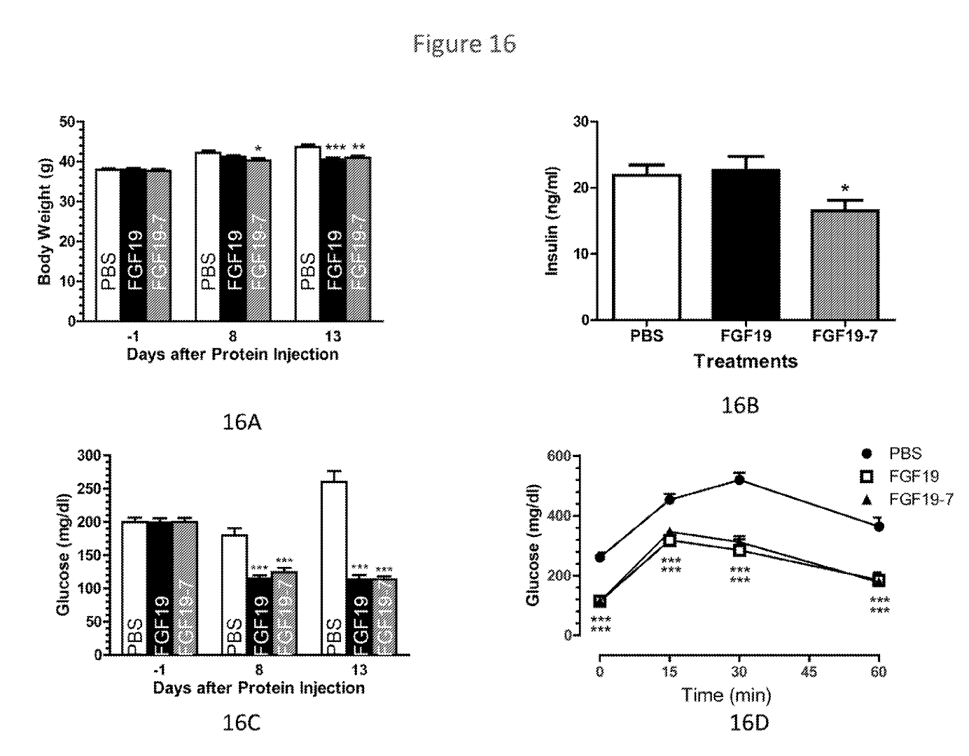

A similar study was also carried out in ob/ob mice, FGF19-7 showed equally efficacy to FGF19 in lowering of fasting plasma glucose levels (FIG. 16) and improvements in OGTT (FIG. 16D). Compare to FGF19, FGF19-7 showed better effects on body weight reduction during the 2 week treatment period (FIG. 16A) and a significant plasma insulin lowering (FIG. 16B) which was not observed for FGF19 group during the study. There results together suggest that the ability of FGF19-7 to regulate glucose and TG metabolism, and induction of body weight reduction were unaffected despite the change in receptor specificity.

### One Year Study

In a related study, FGF19 and FGF19-7 were expressed in DIO mice using AAV-mediated DNA delivery.

Since stable long term expression of up to 1 year has been observed with adeno-associated virus (AAV) gene delivery method, we decided to assess the long term effects of FGF19 and FGF19-7 using AAV as gene delivery vehicle. In addition, in order to obtain information on the metabolic effects of FGF19 and FGF19-7 in this adult on-set model, B6D2F1/J male mice were chosen and were first put on high fat diet at 3-4 weeks old prior to AAV virus injection. The study was carried out for 1 year with periodic measurements of body weight, glucose. At termination, body weight, liver weight, plasma glucose, OGTT, TG, insulin, and FGF levels were also measured.

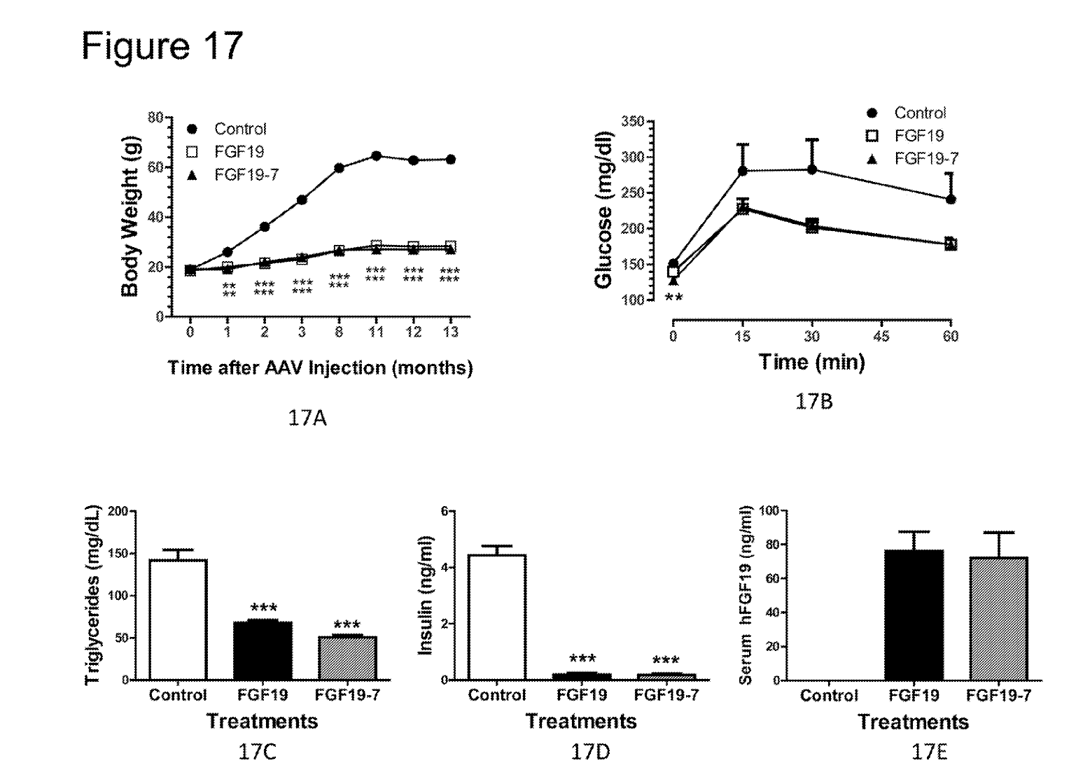

During the course of the 1 year study, mice injected with AAV expression FGF19-7 had reduced body weight gain similar to the group receiving AAV expressing wild type FGF19, suggesting that FGF19 prevented high-fat diet induced obesity in these animals (FIG. 17A). The fasting glucose levels were not significantly different between the groups (data not shown), however, the response of mice receiving AAV virus expressing FGF19 and FGF19-7 to an oral glucose challenge were significantly improved (FIG. 17B). In addition, at termination of the study, both FGF19 and FGF19-7 groups had significantly lower plasma TG and insulin levels to the same degree consistent with the effects observed with the short term studies (FIG. 17) and previously published effects of FGF 19 of glucose regulation.

FIG. 17E highlights that both constructs were expressed at roughly the same level in the DIO mice.

The documents cited herein are incorporated by reference for any purpose.

What is claimed is:

1. A method of identifying a compound that specifically modulates the interaction of FGFR1 and β-Klotho comprising:
(a) determining a baseline level of FGFR1-mediated signaling in a signaling assay system comprising β-Klotho and FGFR1, wherein the FGFR1-mediated signal is one or more of Erk phosphorylation, FGFR1 phosphorylation and FRS2 phosphorylation;
(b) contacting a test compound with the signaling assay system;
(c) detecting a level of FGFR1-mediated signaling in the presence of the test compound; and
(d) comparing the level of FGFR1-mediated signaling in the presence of the test compound with the baseline level of FGFR1-mediated signaling, wherein a difference between the two signaling levels indicates that the test compound modulates the interaction of FGFR1 and β-Klotho.

2. The method of claim 1, wherein the FGFR1 is FGFR1c.

3. The method of claim 1, wherein the FGFR1 is FGFR1b.

4. The method of claim 1, wherein the assay system comprises cells that express β-Klotho and FGFR1.

5. The method of claim 4, wherein the cells are human adipocyte cells.

6. The method of claim 4, wherein the cells are human liver cells.

7. The method of claim 4, wherein the cells are murine 3T3 adipocyte cells.

8. The method of claim 4, wherein the assay system comprises one of a mouse model, a non-human primate model and a rat model.

9. The method of claim 1, wherein the method is performed in the presence of a moiety that, in the presence of FGFR1 and β-Klotho, but in the absence of a test molecule, activates signaling.

10. The method of claim 9, wherein the moiety is one or more of FGF21, FGF19, a mutant form of FGF21, a mutant form of FGF19, an FGF21 analog, a FGF19 analog, an antibody and a peptibody.
